(19) United States
(12) Patent Application Publication
(10) Pub. No.: US 2024/0400603 A1
(43) Pub. Date: Dec. 5, 2024

(54) S- AND SE- ADENOSYL-L-METHIONINE ANALOGUES WITH ACTIVATED GROUPS FOR TRANSFER BY METHYLTRANSFERASES ON TARGET BIOMOLECULES

(71) Applicant: TAGOMICS LIMITED, Coventry (GB)

(72) Inventors: Robert NEELY, Birmingham, West Midlands (GB); Krystian UBYCH, Birmingham, West Midlands (GB); Francisco FERNANDEZ-TRILLO, A Coruña (ES)

(21) Appl. No.: 18/695,485

(22) PCT Filed: Sep. 27, 2022
(86) PCT No.: PCT/GB2022/052438

§ 371 Date: Mar. 26, 2024

(30) Foreign Application Priority Data
Sep. 27, 2021 (GB) 2113794.8

(51) Int. Cl. C07H 19/167; C12N 9/10

(52) U.S. Cl. CPC C07H 19/167; C12N 9/1007

(57) ABSTRACT

Provided herein are analogues of S-adenosyl-L-methionine, methods of their preparation, and complexes and kits comprising the analogues. Said analogues find use in modifying, labelling and analysing a target molecule such as a nucleic acid.

This invention relates to analogues of S-adenosyl-L-methionine, and methods of synthesising said analogues.

### BACKGROUND

S-adenosyl-L-methionine (AdoMet, also known as SAM) is the second most ubiquitous cofactor in human cells, after ATP. It is used as a substrate by a host of enzymes, among which is the methyltransferase family. Methyltransferases are epigenetic regulators that methylate their target molecules (DNA, RNA, proteins or small molecules) using a methyl group from the AdoMet cofactor. The introduction of methyl groups to these classes of target molecules helps to regulate gene expression and normal cellular function. As such, aberrant alterations to the methylation profile of these biomolecules can have detrimental effects and thus can be used as an indicator of disease.

Methyltransferases (MTases) are emerging as important tools for the site-selective modification of DNA, RNA, and proteins. In methyltransferase-directed transfer of activated groups (“mTAG”) labelling, an S-adenosyl-L-methionine cofactor analogue is employed wherein the methyl group of the natural S-adenosyl-L-methionine cofactor is exchanged for a different moiety. A methyltransferase enzyme may then be used to functionalize a target biomolecule with the different moiety using the modified cofactor. By manipulating the chemical structure of the naturally occurring S-adenosyl-L-methionine cofactor, it is possible to use this labelling process as a method for the covalent introduction of functional groups and labels onto biomolecules. mTAG labelling also offers the ability to purify and analyse target biomolecules from cell lysate.

However, there are over 200 methylases in human cells, many of which are likely to interact with any given AdoMet analogue and use this analogue to modify their biological target molecule. As such, it would be beneficial to provide AdoMet analogues which can be used in therapy or in diagnosis. In particular, it would be useful to provide AdoMet analogues which can be tuned to act as a substrate for a particular methyltransferase or subset of methyltransferase enzymes to enhance selectivity for therapeutic or diagnostic reasons.

Current methods for synthesising AdoMet analogues have enabled the introduction of inert modifications, such as benzyl groups, onto the adenine ring. In order to introduce reactive functionalities, protection groups are required which can lower the yield, increase the number of synthetic steps, and limit the functionalities that can be introduced. It is also known to produce AdoMet analogues using an enzyme-catalysed synthetic method. However, this approach relies on compatibility between the enzyme and the functional group being introduced. Another drawback of this approach is the limited scale of the reaction (μmol scale), which is about 1000-fold lower than chemical synthesis.

There is therefore a need for more efficient synthetic methods for the preparation of analogues of AdoMet.

The present disclosure has been devised with these issues in mind.

### BRIEF SUMMARY OF THE DISCLOSURE

Accordingly, there is disclosed a compound of formula (I),

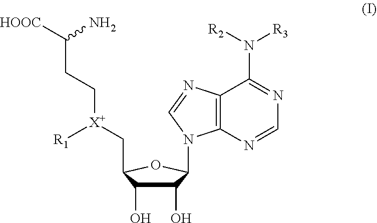

wherein:
X is S or Se;
R₁ has the structure [R₅]_{q}—[L1]ₚ—[HM]ₙ—[L2]ₘ—U—CH₂—; R₂ is H and R₃ is (C₁-C₄) alkyl, (C₂-C₄) alkenyl or (C₂-C₄) alkynyl, with the proviso that R₃ is not propargyl, optionally wherein R₃ is substituted with one or more R₄,
or
R₂ and R₃, together with the nitrogen to which they are attached, form a 5- or 6-membered heterocyclyl ring which is optionally substituted with one or more R₄;
R₄ is selected from the group consisting of: —NRₐR_{b}; —OH; —SH; —CN; —C(O) OR₆; —C(O) R₆; C(O) NRₐR_{b}; N₃; and halo (F, Cl, Br or I);
R₆ is H or unsubstituted C₁₋₄ alkyl;
Rₐ and R_{b} are independently selected from H and unsubstituted (C₁-C₄) alkyl;
L1 is a bond or a linker;
HM is a hydrolysable moiety;
L2 is a linker;
U comprises an unsaturated group selected from an alkene, an alkyne, an aromatic group (e.g. aryl), a carbonyl group, and a sulphur atom comprising one or two S═O bonds; m, n, p and q are each independently selected from 0 and 1; and
R₅ comprises a heavy atom or a heavy atom cluster suitable for phasing of X-ray diffraction data, a radioactive or stable rare isotope, a fluorophore, a fluorescence quencher, an affinity tag, a crosslinking agent, a nucleic acid cleaving reagent, a spin label, a chromophore, a protein, peptide or amino acid which may optionally be modified a nucleotide, nucleoside or nucleic acid which may optionally be modified, a carbohydrate, a lipid, a transfection reagent, an intercalating agent, a nanoparticle or bead, or a functional group,
wherein the functional group is selected from the group consisting of: an amino group (including a protected amino), a thiol group, a 1,2-diol group, a hydrazino group, a hydroxyamino group, a haloacetamide group, a maleimide group, a cyanide group, a cyclic hydrocarbon (such as a bridged cyclic hydrocarbon (e.g. norbornene) or a cycloalkyl group (e.g. a C₃₋₆ cycloalkyl), a halo group (e.g. —F, —Cl, —Br, —I), an aldehyde group, a ketone group, a 1,2-aminothiol group, a azido group, an isothiocyanate or thiocyanate group, an alkene group, such as a terminal alkene, an alkyne group, such as a terminal alkyne group, a 1,3-diene function, a dienophilic function (e.g. an activated carbon-carbon double bond), an arylhalide group, an arylboronic acid group, a terminal haloalkyne group, a terminal silylalkyne group, —N═C═O; —N═C═S, —O—C(O) NH₂, a protected amino, a group comprising a sterically strained alkyne or alkene (such as norbornene or DBCO), a nitrone, a tetrazine, a tetrazole, and 1,2-aminothiol group.

There is further disclosed a method of preparing a compound of formula (I), the method comprising:
(a) reacting a compound of formula (II)

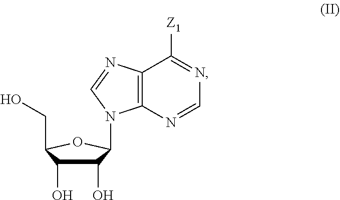

wherein Z₁ is F, Cl, I or Br, with a halogen donor to form a compound of formula (III);

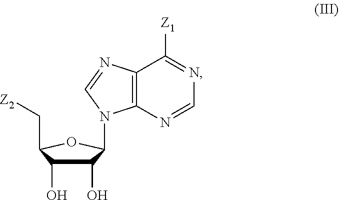

wherein Z₁ and Z₂ are independently selected from F, I, Br and Cl;
(b) reacting the compound of formula (III) with NHR₂R₃, wherein R₂ and R₃ are as defined herein, to form a compound of formula (IV);

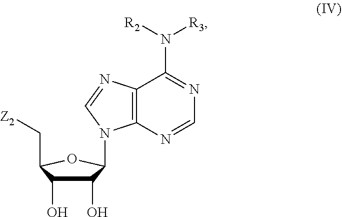

wherein Z₂ is as defined herein,
(c) reacting the compound of formula (IV) with homocysteine (e.g. L-homocysteine) or selenohomocysteine (e.g. L-selenohomocysteine) to form a compound of formula V;

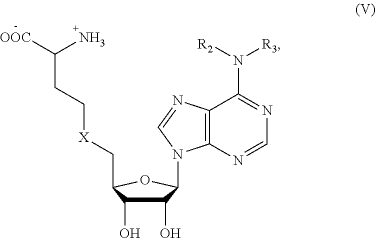

wherein X is Se or S;
and
(d) reacting the compound of formula (V) with R₁-LG to form the compound of formula (I), wherein LG is a leaving group.

In a third aspect, there is disclosed an intermediate of formula (III)

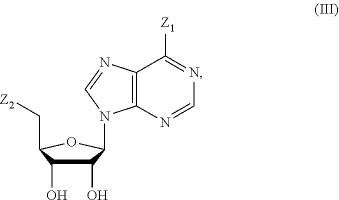

wherein Z₁ and Z₂ are independently selected from F, I, Br and C₁.

There is also provided an intermediate of formula (IV)

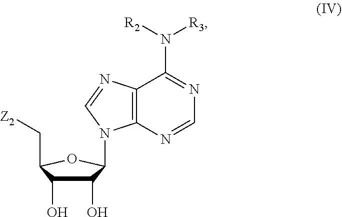

wherein R₂, R₃ and Z₂ are as defined above. Preferably, Z₂ is I.

There is yet further disclosed a complex of a compound of formula (I) and a methyltransferase. The methyltransferase may be capable of using S-adenosyl methionine as a cofactor.

There is also disclosed a composition comprising the compound of formula (I).

There is also disclosed a kit comprising a compound of formula (I), or a composition comprising said compound. The kit may further comprise a methyltransferase, e.g. a methyltransferase capable of using S-adenosyl methionine as a cofactor.

There is also disclosed the use of a compound of formula (I) in a method of modifying a target biomolecule, e.g. a nucleic acid.

There is yet further disclosed a method of modifying a target biomolecule, the method comprising incubating the target biomolecule with a compound of formula (I) and a methyltransferase such that a transferable group (i.e. R₁) of the compound of formula (I) is transferred onto the target biomolecule.

A further aspect of the disclosure comprises a biomolecule (e.g. DNA or RNA molecule or base or fraction thereof or protein or amino acid or peptide or polypeptide thereof) having bonded thereto a molecule R₁, wherein
R₁ has the structure [R₅]_{q}—[L1]ₚ—[HM]ₙ—[L2]ₘ—U—CH₂—
L1 is a bond or a linker;
HM is a hydrolysable moiety;
L2 is a linker;
U comprises an unsaturated group selected from an alkene, an alkyne, an aromatic group (e.g. aryl), a carbonyl group, and a sulphur atom comprising one or two S═O bonds;
m, n, p and q are each independently selected from 0 and 1; and
R₅ comprises a heavy atom or a heavy atom cluster suitable for phasing of X-ray diffraction data, a radioactive or stable rare isotope, a fluorophore, a fluorescence quencher, an affinity tag, a crosslinking agent, a nucleic acid cleaving reagent, a spin label, a chromophore, a protein, peptide or amino acid which may optionally be modified a nucleotide, nucleoside or nucleic acid which may optionally be modified, a carbohydrate, a lipid, a transfection reagent, an intercalating agent, a nanoparticle or bead, or a functional group,
wherein the functional group is selected from the group consisting of: an amino group (including a protected amino), a thiol group, a 1,2-diol group, a hydrazino group, a hydroxyamino group, a haloacetamide group, a maleimide group, a cyanide group, a cyclic hydrocarbon (such as a bridged cyclic hydrocarbon (e.g. norbornene) or a cycloalkyl group (e.g. a C₃₋₆ cycloalkyl), a halo group (e.g. —F, —Cl, —Br, —I), an aldehyde group, a ketone group, a 1,2-aminothiol group, a azido group, an isothiocyanate or thiocyanate group, an alkene group, such as a terminal alkene, an alkyne group, such as a terminal alkyne group, a 1,3-diene function, a dienophilic function (e.g. an activated carbon-carbon double bond), an arylhalide group, an arylboronic acid group, a terminal haloalkyne group, a terminal silylalkyne group, —N═C═O; —N═C═S, —O—C(O)NH₂, a protected amino, a group comprising a sterically strained alkyne or alkene (such as norbornene or DBCO),, a nitrone, a tetrazine, a tetrazole, and 1,2-aminothiol group.

### DETAILED DESCRIPTION

The disclosure will now be described by way of example and with reference to the accompanying Figures, in which:

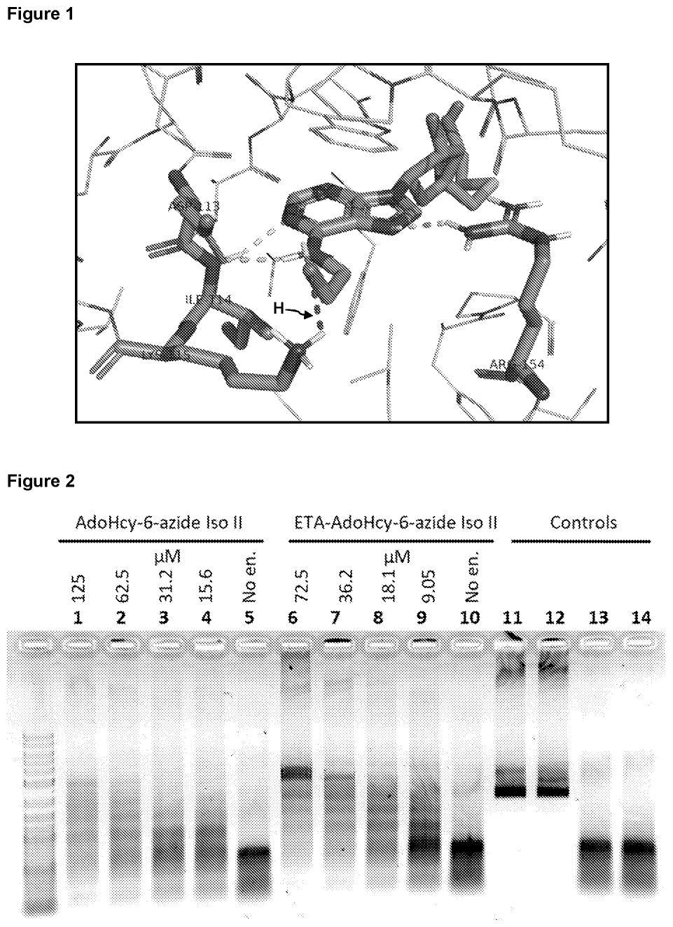

FIG. 1 is a model of the cofactor analogue AdoHcy-ETA bound to M.Mpel, generated using the crystal structure of AdoHyc bound to M.Mpel. The dashed line ‘H’ indicates a hydrogen bond between the oxygen of the hydroxy group of the cofactor and the ammonium group of LYS-115;

FIG. 2 shows the bands obtained on an agarose gel following a restriction assay of pUC19 following incubation with M.Mpel and cofactors according to an embodiment of the invention;

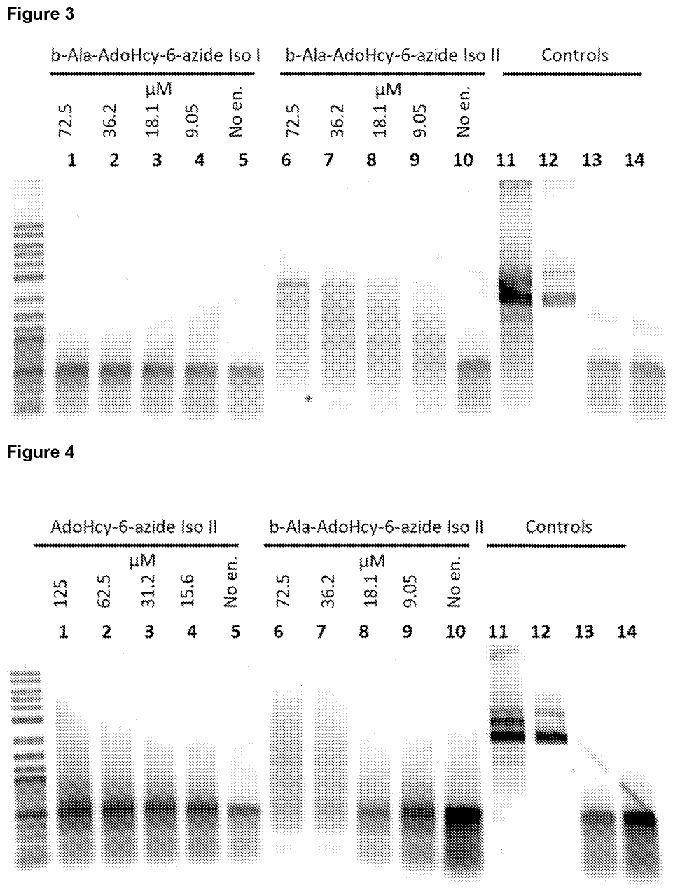

FIG. 3 shows the bands obtained on an agarose gel following a restriction assay of pUC19 following incubation with M.Mpel and cofactors according to a further embodiment of the invention;

FIG. 4 shows the bands obtained on an agarose gel following a restriction assay of pUC19 following incubation with M.Mpel and cofactors according to another embodiment of the invention; and

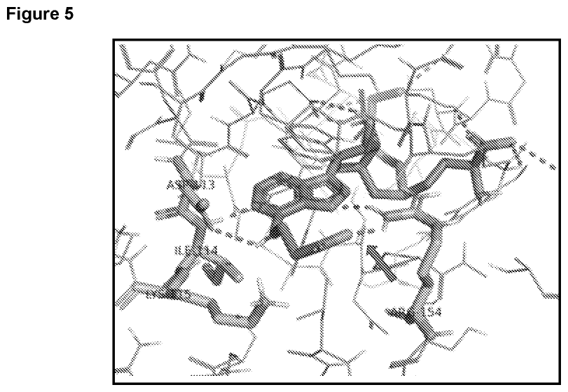

FIG. 5 is a model showing interactions between the cofactor analogue b-Ala-AdoHcy-6-azide and residues of the M.Mpel protein, generated using the crystal structure of AdoHcy bound to M.Mpel. The dashed lines indicate potential hydrogen bonding interactions between the cofactor analogue and the surrounding protein amino acids. A favourable electrostatic interaction is predicted between ARG-154 and the carboxylic acid group appended to the b-Ala-AdoHcy-6-azide cofactor analogue.

### DEFINITIONS

Unless otherwise stated, the following terms used in the specification and claims have the following meanings set out below.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

The term “halo” or “halogen” refers to one of the halogens, group 17 of the periodic table. In particular the term refers to fluorine, chlorine, bromine and iodine.

The term “(C₁-C₄) alkyl” refers to a linear or branched hydrocarbon chain containing 1, 2, 3 or 4 carbon atoms, for example methyl, ethyl, n-propyl, iso-propyl, n-butyl or iso-butyl. “C₁₋₆ alkyl” and “C₁₋₁₀ alkyl” similarly refer to such groups containing up to 6 or up to 10 carbon atoms, respectively. Alkylene groups are divalent alkyl groups and may likewise be linear or branched and have two points of attachment to the remainder of the molecule.

Furthermore, an alkylene group may, for example, correspond to one of those alkyl groups listed in this paragraph. For example, C₁₋₄ alkylene may be —CH₂—, —CH₂CH₂—, —CH₂CH(CH₃)—, —CH₂CH₂CH₂— or —CH₂CH(CH₃)CH₂—. The alkyl and alkylene groups may be unsubstituted or substituted by one or more substituents. Possible substituents are described herein.

The term “(C₂-C₄) alkenyl” refers to a branched or linear hydrocarbon chain containing at least one double bond and having 2, 3 or 4 carbon atoms. The double bond(s) may be present as the E or Z isomer. The double bond may be at any possible position of the hydrocarbon chain. For example, the “(C₂-C₄) alkenyl” may be ethenyl, propenyl, butenyl or butadienyl. Alkenylene groups are divalent alkenyl groups and may likewise be linear or branched and have two points of attachment to the remainder of the molecule. Furthermore, an alkenylene group may, for example, correspond to one of those alkenyl groups listed in this paragraph. For example, alkenylene may be —CH═CH—, —CH₂CH═CH—, —CH(CH₃) CH═CH— or —CH₂CH═CH—. Alkenyl and alkenylene groups may unsubstituted or substituted by one or more substituents. Possible substituents are described herein.

The term “(C₂-C₄) alkynyl” includes a branched or linear hydrocarbon chain containing at least one triple bond and having 2, 3 or 4 carbon atoms. The triple bond may be at any possible position of the hydrocarbon chain. For example, the “(C₂-C₄) alkynyl” may be ethynyl, propynyl or butynyl. Alkynylene groups are divalent alkynyl groups and may likewise be linear or branched and have two points of attachment to the remainder of the molecule. Furthermore, an alkynylene group may, for example, correspond to one of those alkynyl groups listed in this paragraph. For example, alkynylene may be —C═C—, —CH₂C═C— or —CH₂C═CCH₂—. Alkynyl and alkynylene groups may unsubstituted or substituted by one or more substituents. Possible substituents are described herein. For example, substituents may be those described above as substituents for alkyl groups.

As used herein, a 5- or 6-membered “heterocyclyl”, “heterocyclic” or “heterocycle” group includes a non-aromatic saturated or partially saturated monocyclic system. Monocyclic heterocyclic rings may contain 1, 2 or 3 heteroatoms independently selected from nitrogen, oxygen or sulfur in the ring, including or in addition to the nitrogen which attaches the ring to the rest of the molecule. By partially saturated it is meant that the ring may comprise one or two double bonds. The double bond will typically be between two carbon atoms but may be between a carbon atom and a nitrogen atom. Heterocycles comprising at least one nitrogen include, for example, pyrrolidinyl, piperidinyl, piperazinyl, morpholinyl, thiomorpholinyl, tetrahydropyrazolyl, tetrahydropyridinyl, and the like. As the skilled person would appreciate, any heterocycle may be linked to another group via any suitable atom, such as via a carbon or nitrogen atom.

The term “aromatic” when applied to a substituent as a whole includes a single ring or polycyclic ring system with 4n+2 electrons in a conjugated IT system within the ring or ring system where all atoms contributing to the conjugated IT system are in the same plane.

The term “aryl” includes an aromatic hydrocarbon ring system. The ring system has 4n+2 electrons in a conjugated IT system within a ring where all atoms contributing to the conjugated IT system are in the same plane. For example, the “aryl” may be phenyl and naphthyl. The aryl system itself may be substituted with other groups.

The term “carbonyl” refers to a functional group comprising a carbon atom with a double bond to an oxygen atom. The group includes aldehydes (—C(O)H); ketones (—C(O)R); carboxylic acids (—C(O)OH); esters (—C(O)OR), amides (—C(O)NR′R″), enones (—C(O)C(R)CR′R″), acyl halides (—C(O)X), acid anhydrides (—C(O)OC(O)R) and imides (—C(O)N(R)C(O)R′).

A bond terminating in a “or” represents that the bond is connected to another atom that is not shown in the structure. A bond terminating inside a cyclic structure and not terminating at an atom of the ring structure represents that the bond may be connected to any of the atoms in the ring structure where allowed by valency.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Where a compound of the invention has two or more stereo centres any combination of (R) and(S) stereoisomers is contemplated. The combination of (R) and(S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%, for example at least 90%, at least 95% or at least 99%.

The compounds of this disclosure may possess one or more asymmetric centres; such compounds can therefore be produced as individual (R)- or(S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess MASTL inhibitory activity.

Z/E (e.g. cis/trans) isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high-pressure liquid chromatography (HPLC). Thus, chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and for specific examples, 0 to 5% by volume of an alkylamine e.g. 0.1% diethylamine. Concentration of the eluate affords the enriched mixture.

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

Compounds and salts described in this specification may be isotopically-labelled (or “radio-labelled”). Accordingly, one or more atoms are replaced by an atom having an atomic mass or mass number different from the atomic mass or mass number typically found in nature. Examples of radionuclides that may be incorporated include ²H (also written as “D” for deuterium), ³H (also written as “T” for tritium), ¹¹C, ¹³C, ¹⁴C, ¹⁵O, ¹⁷O, ¹⁸O, ¹³N, ¹⁵N, ¹⁸F, ³⁶Cl, ¹²³I, ²⁵I, ³²p, ³⁵S and the like. The radionuclide that is used will depend on the specific application of that radio-labelled derivative. For example, for in-vitro competition assays, ³H or ¹⁴C are often useful. For radio-imaging applications, ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F.

Isotopically-labelled compounds can generally be prepared by conventional techniques known to those skilled in the art or by processes analogous to those described using an appropriate isotopically-labelled reagent in place of the non-labelled reagent previously employed.

### Compounds

The present disclosure provides a compound of formula (I),

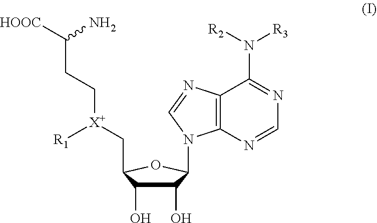

wherein:
X is S or Se
R₁ has the structure [R₅]_{q}—[L1]ₚ—[HM]ₙ—[L2]ₘ—U—CH₂—;
R₂ is H and R₃ is (C₁-C₄) alkyl, (C₂-C₄) alkenyl or (C₁-C₄) alkynyl, with the proviso that R₃ is not propargyl, optionally wherein R₃ is substituted with one or more R₄,
or
R₂ and R₃, together with the nitrogen to which they are attached, form a 5- or 6-membered heterocyclyl ring which is optionally substituted with one or more R₄;
R₄ is selected from the group consisting of: —NRₐR_{b}; —OH; —SH; —C(O)OR₆; —C(O)R₆; C(O) NRₐR_{b}; N₃; and halo (F, Cl, Br or I);
Rₐ and R_{b} are independently selected from H and (C₁-C₄) alkyl;
R₆ is H or C₁₋₄ alkyl;
L1 is a bond or a linker;
HM is a hydrolysable moiety;
L2 is a linker;
U comprises an unsaturated group selected from an alkene, an alkyne, an aromatic group (e.g. aryl), a carbonyl group, and a sulphur atom comprising one or two S═O bonds;
m, n, p and q are each independently selected from 0 and 1;
R₅ comprises or consists of a heavy atom or a heavy atom cluster suitable for phasing of X-ray diffraction data, a radioactive or stable rare isotope, a fluorophore, a fluorescence quencher, an affinity tag, a crosslinking agent, a nucleic acid cleaving reagent, a spin label, a chromophore, a protein, peptide or amino acid which may optionally be modified a nucleotide, nucleoside or nucleic acid which may optionally be modified, a carbohydrate, a lipid, a transfection reagent, an intercalating agent, a nanoparticle or bead, or a functional group,
wherein the functional group is selected from the group consisting of: an amino group (including a protected amino group), a thiol group, a 1,2-diol group, a hydrazino group, a hydroxyamino group, a haloacetamide group, a maleimide group, a cyanide group, a cyclic hydrocarbon (such as a bridged cyclic hydrocarbon or a cycloalkyl group (e.g. a C₃₋₆ cycloalkyl), a halo group (e.g. —F, —Cl, —Br, —I), an aldehyde group, a ketone group, a 1,2-aminothiol group, an azido group, an isothiocyanate or thiocyanate group, an alkene group, such as a terminal alkene, an alkyne group, such as a terminal alkyne group, a 1,3-diene function, a dienophilic function (e.g. an activated carbon-carbon double bond), an arylhalide group, an arylboronic acid group, a terminal haloalkyne group, a terminal silylalkyne group, —N═C═O; —N═C═S, —O—C(O)NH₂, a group comprising a sterically strained alkyne or alkene (such as norbornene or DBCO), a nitrone, a tetrazine or a tetrazide.

In some embodiments X is Se. In some embodiments, X is S. Preferably, X is S.

In some embodiments, m and n are both 1. In some embodiments, m is 1 and n is 0. In some embodiments m is 0 and n is 1.

In some embodiments, p is 1.

In some embodiments, q is 1.

In some embodiments, m and n are both 0. In some embodiments, m and n are both 0, and p and q are both 1. In some embodiments, m, n and p are all 0, and q is 1. In some embodiments, m, n, p and q are all 0. In some embodiments, m, n, p and q are all 1.

Thus, in some embodiments, R₁ has the structure: [R₅]—[L1]—[HM]—[L₂]—U—CH₂—, wherein R₅, L₁, HM, L₂ and U are as defined herein.

In some embodiments, R₁ has the structure: R₅-L₁-U—CH₂, wherein R₅, L₁ and U are as defined herein.

In some embodiments, R₁ has the structure: R₅—U—CH₂, wherein R₅ and U are as defined herein.

In some embodiments, R₁ has the structure: U—CH₂, wherein U is as defined herein.

In some embodiments, R₅ comprises a heavy atom or a heavy atom cluster suitable for phasing of X-ray diffraction data, a radioactive or stable rare isotope, a fluorophore, a fluorescence quencher, an affinity tag, a crosslinking agent, a nucleic acid cleaving reagent, a spin label, a chromophore, a protein, peptide or amino acid which may optionally be modified, a nucleotide, nucleoside or nucleic acid which may optionally be modified, a carbohydrate, a lipid, a transfection reagent, an intercalating agent, a nanoparticle or bead, or a functional group.

In some embodiments, R₅ comprises a heavy atom or a heavy atom cluster suitable for phasing of X-ray diffraction data. The heavy atom or heavy atom cluster suitable for phasing of X-ray diffraction data may be selected from copper, zinc, selenium, bromine, iodine, ruthenium, palladium, cadmium, tungsten, platinum, gold, mercury, bismuth, samarium, europium, terbium, uranium, Ta₆Br₁₄, and Fe₄S₄.

In some embodiments, R₅ comprises a radioactive or stable rare isotope. In some embodiments, the radioactive rare isotope is ¹⁹F or ¹²⁷I. In some embodiments, the stable rare isotope is ³H (T), ¹⁴C, ³²P, ³³P, ³⁵S, ¹²⁵I, ¹³I, ²H (D), ¹³C, ¹⁵N, ¹⁷O or ¹⁸O.

In some embodiments, R₅ comprises a fluorophore. The fluorophore may be Alexa, BODIPY, bimane, coumarin, Cascade blue, dansyl, dapoxyl, fluorescein, mansyl, MANT, Oregon green, pyrene, rhodamine, Texas red, TNS, fluorescent nanocrystals (quantum dots), oxazine, Atto, or a cyanine fluorophore.

In some embodiments, R₅ comprises a fluorescence quencher. Suitable fluorescence quenchers include dabcyl, QSY and BHQ.

In some embodiments, R₅ comprises an affinity tag. In some embodiments, the affinity tag is a peptide tag (e.g. a his-tag, a strep-tag, a flag-tag, a c-myc-tag, a HA-tag, an epitope or glutathione), a metal-chelating group (e.g. nitrilotriacetic acid, ethylenediaminetetraacecetic acid (EDTA), 1,10-pehnanthroline, a crown ether or a HiS4-8 peptide) an isotope coded affinity tag, biotin, maltose, mannose, glucose, N-acetylglucosamine, N-acetylneuraminic acid, galactose, N-acetylgalactosamine, digoxygenin or dinitrophenol.

In some embodiments, R₅ comprises a cross-linking agent. Suitable cross-linking agents include mono- or bifunctional platinum (II) complexes, maleimides, iodacetamides, aldehydes and photocrosslinking agents such as arylazide, a diazo compound, a 2-nitrophenyl compound, psoralen and a benzophenone compound.

In some embodiments, R₅ comprises a nucleic acid cleaving reagent. Suitable nucleic acid cleaving reagents include iron-EDTA, copper-1,10-phenanthroline, acridine or a derivative thereof, an enediyne compound and a rhodium complex.

In some embodiments, R₅ comprises a spin label. In some embodiments, the spin label is 2,2,6,6,-tetramethyl-piperidin-1-oxyl or 2,2,5,5,-tetramethyl-pyrrolidin-1-oxyl.

In some embodiments, R₅ comprises a chromophore.

In some embodiments, R₅ comprises a protein, peptide or amino acid which may optionally be modified. An amino acid modifications include B- and y-amino acids. In some embodiments, a peptide modification is selected from the group consisting of depsipeptides, vinylogous peptides, permethylated peptides, peptoids, azapeptides (azatides), oligocarbamates, oligoureas, oligosulfones, oligosulfonamides, oligosulfinamides, pyrrole-imidazole-hydroxypyrrole polyamides and peptide nucleic acids (PNA).

In some embodiments, R₅ comprises a nucleotide, nucleoside or nucleic acid which may optionally be modified. In some embodiments, R₅ is a modified nucleic acid, such as a peptide nucleic acid (PNA), a locked nucleic acid (LNA) or a phosphorothioate modified nucleic acids.

In some embodiments, R₅ comprises a carbohydrate or a lipid (e.g. cholesterol).

In some embodiments, R₅ comprises a transfection reagent. Suitable transfection reagents include cationic lipids (e.g. Lipofectamin and derivatives commercially available from Invitrogen, CA, USA), cationic polymers (e.g. polyethyleneimine (PEI) commercially available from Sigma) and polycationic dendrimers.

In some embodiments, R₅ comprises an intercalating agent. Intercalating agents are typically planar or near planar aromatic ring systems which are able to bind between neighbouring base-pairs in double-stranded nucleic acids. Suitable intercalating agents are include ethidium, thiazole orange, acridine or a derivative thereof, and pyrene.

In some embodiments, R₅ comprises a nanoparticle or bead. Suitable nanoparticles include gold and silver clusters. Suitable beads include silica beads, magnetic beads and polystyrene microspheres (e.g. commercially available from Molecular Probes, OR, USA).

In some embodiments, R₅ comprises or consists of a functional group selected from the group consisting of: an amino group (including a protected amino group), a thiol group, a 1,2-diol group, a hydrazino group, a hydroxyamino group, a haloacetamide group, a maleimide group, a cyanide group, a cyclic hydrocarbon (such as a bridged cyclic hydrocarbon (e.g. norbornene) or a cycloalkyl group (e.g. a C₃₋₆ cycloalkyl)), a halo group (e.g. —F, —Cl, —Br, —I), an aldehyde group, a ketone group, a 1,2-aminothiol group, an azido group, an isothiocyanate or thiocyanate group, an alkene group, such as a terminal alkene, an alkyne group, such as a terminal alkyne group, a 1,3-diene function, a dienophilic function (e.g. an activated carbon-carbon double bond), an arylhalide group, an arylboronic acid group, a terminal haloalkyne group, a terminal silylalkyne group, —N═C═O; —N═C═S, —O—C(O)NH₂, a group comprising a sterically strained alkyne or alkene (such as norbornene or DBCO), a nitrone, a tetrazine or a tetrazole.

In some embodiments, R₅ comprises or is halo (—F, —Cl, —Br, —I); —C═C; —C═C; —N₃; —N═C═O; —N═C═S; —O—C(O)NH₂; —SH; epoxide; —NH₂; —CEN; a nitrone, a tetrazine, a tetrazole or a group comprising a sterically strained alkyne or alkene. Sterically strained alkynes or alkenes are found in moieties such as norbornene, cyclooctynes (e.g. dibenzylcyclooctyne (DBCO), difluorocyclooctyne, biarylazacyclooctynone), and (trans) cycloalkenes. In some embodiments, R₅ comprises or is norbornene or a cyclooctyne. In some embodiments, R₅ comprises or is DBCO

In some embodiments, R₅ is —N₃.

L₁ may be a linker comprising a linear chain of from 1 to 50, from 2 to 40, from 3 to 30, from 4 to 20 or from 5 to 15 atoms, for example carbon, oxygen and/or nitrogen atoms.

In some embodiments, L₁ comprises a hydrocarbon (e.g. an alkyl) and/or a polyether chain. Additionally or alternatively, L₁ may comprise an aryl moiety, for example a C₆H₄ arene ring. Additionally or alternatively, L₁ may comprise an aromatic group, e.g. a C₆H₄ (C═O) NH group.

In some embodiments, L₁ comprises a linear C₁-C₁₀ alkyl chain, e.g. a C₂-C₈ or a C₃-C₆ alkyl chain. In some embodiments, the alkyl chain is unsubstituted. In some embodiments, L₁ is a C₃ alkylene group, preferably unsubstituted.

In some embodiments, L₁ comprises a polyether chain. In some embodiments, L₁ comprises a polyethylene glycol chain. The polyethylene glycol chain may comprise up to 15, or up to 10 monomers of ethylene glycol, e.g. 9, 8, 7, 6, 5, 4, 3, 2, or 1 monomers of ethylene glycol. In some embodiments, the polyethylene glycol chain comprises from 1 to 5 or from 2 to 3 monomers of ethylene glycol.

In some embodiments, L₁ has the structure:

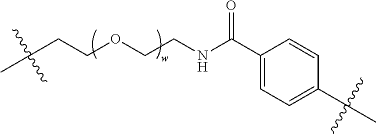

wherein w is an integer of from 1 to 15, e.g. from 2 to 10 or from 3 to 5. In some embodiments, w is 2 or 3.

In some embodiments, U is —C═C— or —C═C—. Preferably, U is —C═C—.

L₂ may be a linker comprising a linear chain of from 1 to 20, from 2 to 15, from 3 to 10 or from 4 to 9 atoms (e.g. carbon, oxygen and/or nitrogen atoms). The linker may be substituted or unsubstituted. In some embodiments, L₂ comprises a hydrocarbon (e.g. an alkyl) chain. In some embodiments, L₂ comprises a linear C₁-C₁₀ alkyl chain, e.g. a C₂-C₈ or a C₄-C₆ alkyl chain. In some embodiments the alkyl chain is unsubstituted. In some embodiments the alkyl chain is substituted. In some embodiments, L₂ is a linear, unsubstituted C₂, C₃ or C₄ alkyl chain, preferably a C₄ alkyl chain (i.e. —CH₂CH₂CH₂CH₂—, butylene).

The hydrolysable moiety (HM) may be selected from the group consisting of:

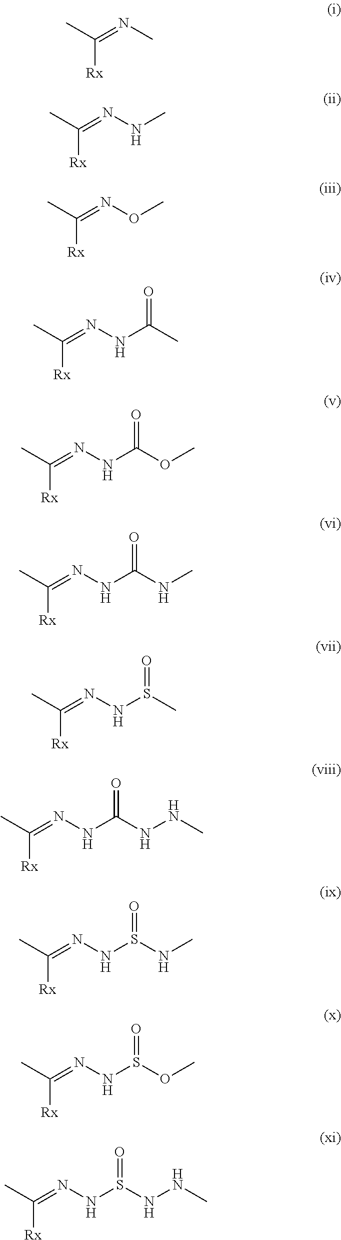

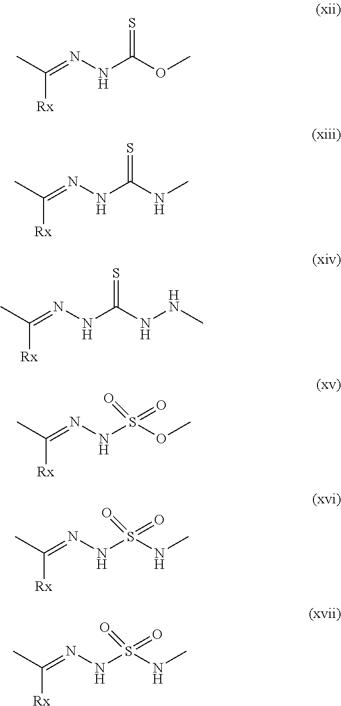

wherein Rx is selected from: a hydrogen atom, a deuterium atom and unsubstituted C₁-C₄ alkyl (e.g. CH₃).

The hydrolysable moiety may be a Schiff base, for example, an imine moiety, an oxime moiety and/or a hydrazone moiety.

In some embodiments, the hydrolysable moiety comprises a disulphide (S-S) bond.

In some embodiments, the hydrolysable moiety has the structure:

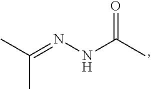

wherein Rx is as defined above.

In some embodiments, R₁ has the structure:

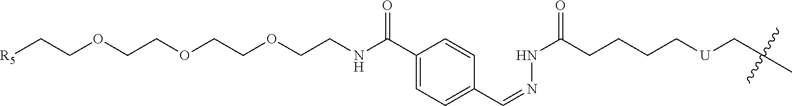

In some embodiments, R₁ has the structure:

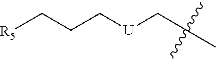

In some embodiments, R₁ has the structure:

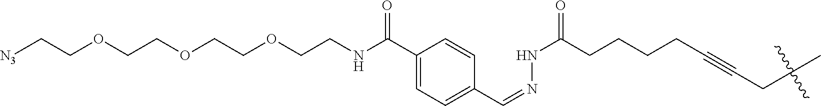 [SMILES: *CC#CCCCCC(=O)N/N=C\c1ccc(C(=O)NCCOCCOCCOCCN=[N+]=[N-])cc1]

In some embodiments, R₁ has the structure:

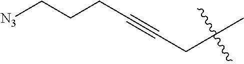 [SMILES: *CC#CCCCN=[N+]=[N-]]

In some embodiments, R₁ has the structure:

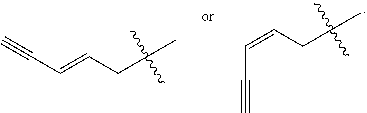

R₃ is C₁-C₄ alkyl, C₂-C₄ alkenyl or C₂-C₄ alkynyl, with the proviso that R₃ is not propargyl. The C₁-C₄ alkyl, C₂-C₄ alkenyl or C₂-C₄ alkynyl group may be substituted or unsubstituted. In some embodiments, R₃ is C₂-C₄ or C₂-C₃ alkenyl. In some embodiments, R₃ is C₁-C₄ alkyl or C₂-C₃ alkyl. In some embodiments R₃ is substituted with one or more R₄. In some embodiments R₃ is substituted with one R₄. In some embodiments R₃ is C₁-C₄ alkyl substituted with one R₄. In some embodiments, R₃ is not unsubstituted C₁-C₄ or C₁-C₂ alkyl. In some embodiments R₃ is not unsubstituted methyl.

In some embodiments, R₂ is H and R₅ is (C₁-C₄) alkyl substituted with one or more R₄, optionally wherein R₃ is substituted with one R₄. In some embodiments R₂ is H and R₃ is C₂ alkyl substituted with one or more R₄, optionally wherein R₃ is substituted with one R₄.

R₄ is selected from the group consisting of: —NRₐR_{b}; —OH; —SH; —C(O)OR₆; —C(O) R₆; —C(O)CH₃; —C(O)OCH₃; C(O)NRₐR_{b}; N₃; and halo (F, Cl, Br or I), wherein Rₐ and R_{b} are independently selected from H and (C₁-C₄) alkyl, and wherein R₆ is H or C₁₋₄ alkyl;. In some embodiments, Rₐ and R_{b} are both H. In some embodiments, one of Rₐ and R_{b} is H, and the other is CH₃. In some embodiments, Re is H. In some embodiments, R₆ is C₁₋₄ alkyl. In some embodiments, R₆ is CH₃.

In some embodiments, R₄ is selected from the group consisting of: —NH₂; —OH; —C(O)OH; N₃; and halo (preferably Cl or F). In some embodiments R₄ is selected from: —NRₐR_{b}; —OH; —SH; —C(O) OR₆; —C(O) R₆; and C(O)NRₐR_{b}. In some embodiments, R₄ is —OH. In some embodiments, R₄ is —C(O)OH.

In some embodiments, R₂ is H and R₃ is selected from:

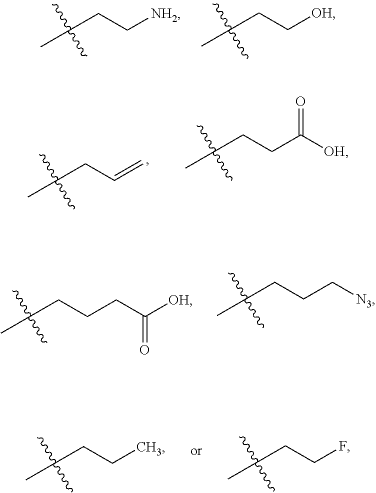

Alternatively, R₂ and R₃, together with the nitrogen to which they are attached, a 5- or 6-membered heterocyclyl ring which is optionally substituted with one or more R₄. The heterocyclyl ring may be selected from pyrrolidinyl, piperidinyl, piperazinyl, morpholinyl, thiomorpholinyl, tetrahydropyrazolyl, and tetrahydropyridinyl. In some embodiments, the heterocyclyl ring is pyrrolidinyl.

In some embodiments, R₂ and R₃, together with the nitrogen to which they are attached, may form the structure:

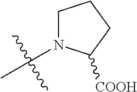 [SMILES: *N1CCCC1C(=O)O]

The structure may be

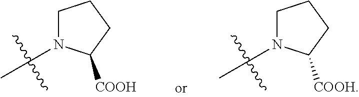 [SMILES: *N1CCC[C@H]1C(=O)O ; *N1CCC[C@@H]1C(=O)O]

In some embodiments the structure is

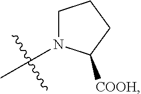 [SMILES: *N1CCC[C@H]1C(=O)O]

In some embodiments, R₂ is H and R₃ is:

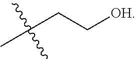 [SMILES: *CCO]

In some embodiments, each of R₁, R₂, R₃, R₄, R₅, L₁, L₂, HM, U, m, n, p and q have any of the meanings defined in any of paragraphs (1) to (22) hereinafter:
(1) R₅ is azide.
(2) U comprises or is an alkyne, optionally U is —C═C—.
(3) U comprises or is an alkene, optionally U is —C═C—.
(4) R₅ is azide and U is —C═C—.
(5) R₅ is azide and U is —C═C—.
(6) L₁ comprises a linear C₁₋₁₀ alkyl chain, optionally wherein the chain is unsubstituted.
(7) L₁ comprises a polyethylene glycol chain, optionally wherein the polyethylene glycol chain comprises up to 15, or up to 10 monomers of ethylene glycol.
(8) R₅ and U are defined in any one of paragraphs (1) to (5) above, and L₁ comprises a linear C₁₋₁₀ alkyl chain, optionally wherein the chain is unsubstituted.
(9) R₅ and U are defined in any one of paragraphs (1) to (5) above, and L₁ comprises a C₃ alkyl chain, optionally unsubstituted.
(10) R₅ and U are defined in any one of paragraphs (1) to (5) above, and L₁ comprises a polyethylene glycol chain, optionally wherein the polyethylene glycol chain comprises up to 15, or up to 10 monomers of ethylene glycol. Preferably the polyethylene glycol chain comprises from 1 to 5 or from 2 to 3 monomers of ethylene glycol.
(11) R₅, U and L₁ are defined in any one of paragraphs (1) to (10) above, and L₁ further comprises an aryl moiety, for example a C₆H₄ arene ring, or an aromatic group, e.g. a C₆H₄ (C═O) NH group.
(12) R₅, U and L₁ are defined in any one of paragraphs (1) to (11) above, and m and n are both 1.
(13) R₅, U and L₁ are defined in any one of paragraphs (1) to (11) above, and m and n are both 0.
(14) HM is a Schiff base, optionally having the structure:

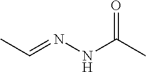

(15) R₅, U, L₁, m and n are defined in any one of paragraphs (1) to (13) above, and HM is defined in paragraph (12) above.
(16) HM is a defined by paragraph (14) above and L₂ comprises a linear C₁₋₁₀ alkyl chain, e.g. a C₂₋₈ or a C₄₋₆ alkyl chain, optionally wherein L₂ is a linear, unsubstituted C₂, C₃ or C₄ alkyl chain.
(17) R₅, U, L₁, m and n are defined in any one of paragraphs (1) to (13) above, wherein HM and L₂ are defined in paragraph (16) above.
(18) R₂ is H and R₃ is C₁₋₄ alkyl, optionally substituted with one R₄ selected from —NRₐR_{b}; —OH; —SH; —C(O) OH; —C(O) H; —C(O) CH₃; —C(O) OCH₃; C(O)NRₐR_{b}; N₃; and halo (F, Cl, Br or I), wherein Rₐ and R_{b} are as defined herein.
(19) R₂ is H and R₃ is C₁₋₄ alkyl substituted with one R₄ selected from —NH₂; —OH; —C(O) OH; N₃; and halo.
(20) R₅, U, L₁, m, n, HM and L₂ are defined in any one of paragraphs (1) to (17) above, R₂ is H and R₃ is C₁₋₄ alkyl substituted with one R₄ selected from —NH₂; —OH; —C(O) OH; N₃; and halo.
(21) R₅, U, L₁, m, n, HM and L₂ are defined in any one of paragraphs (1) to (17) above, R₂ is H and R₃ is C₁₋₄ alkyl substituted with one R₄, wherein R₄ is —OH.
(22) R₅, U, L₁, m, n, HM and L₂ are defined in any one of paragraphs (1) to (17) above, and R₂ and R₃, together with the nitrogen to which they are attached, form the structure:

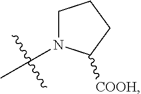 [SMILES: *N1CCCC1C(=O)O]

optionally wherein the structure is

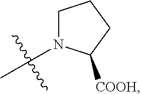 [SMILES: *N1CCC[C@H]1C(=O)O]

(23) R₁, R₂, R₃, R₄, R₅, L₁, L₂, HM, U, m and n have any of the meanings defined in any of paragraphs (1) to (22) above, and X is Se.
(24) R₁, R₂, R₃, R₄, R₅, L₁, L₂, HM, U, m and n have any of the meanings defined in any of paragraphs (1) to (22) above, and X is S.

In some embodiments, the compound has a structure selected from those listed in Table 1.

TABLE 1
Structures of AdoMet analogues

| Structure | Name |
|---|---|
| 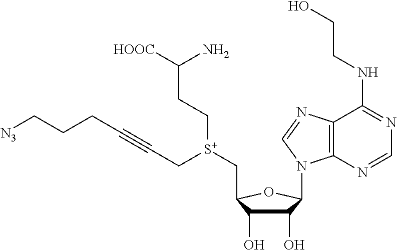 | ETA- AdoHcy- 6-azide |
| 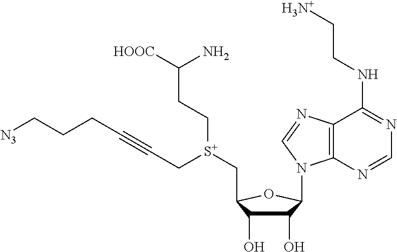 | EDA- AdoHcy- 6-azide |
| 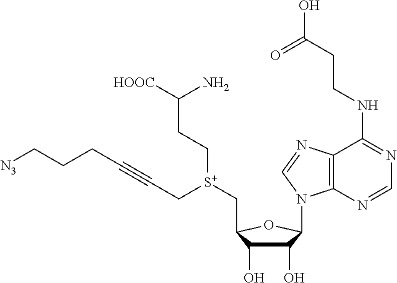 | b-Ala- AdoHcy- 6-azide |
| 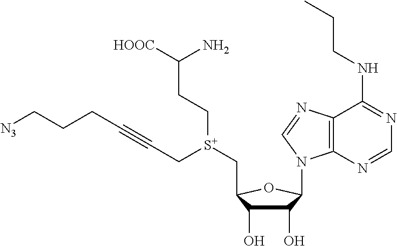 | Propyl- AdoHcy- 6-azide |
| 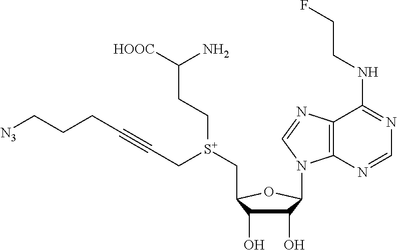 | Fluoro- AdoHcy- 6-azide |
| 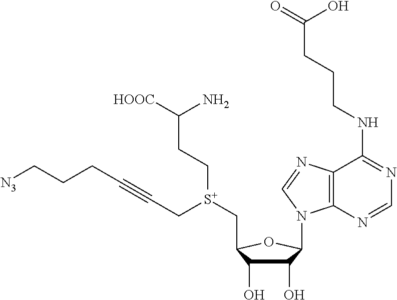 | GABA- AdoHcy- 6-azide |
| 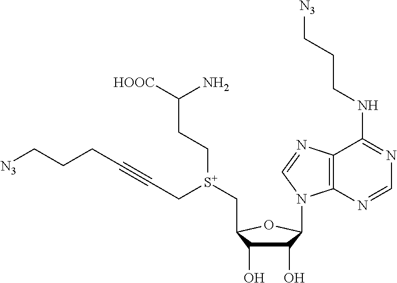 | PAA- AdoHcy- 6-azide |
| 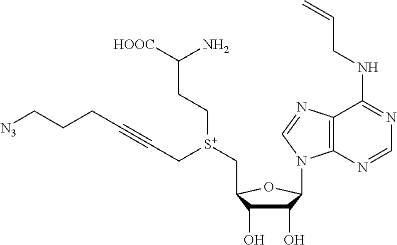 | Allyl- AdoHcy- 6-azide |
| 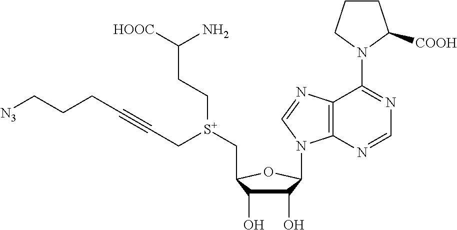 | Pro- AdoHcy- 6-azide |
| 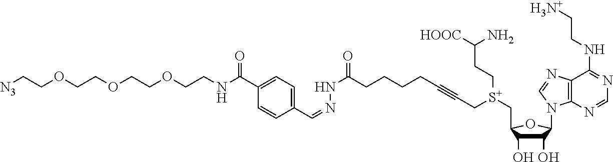 | EDA- AdoHcy- Hydr |
| 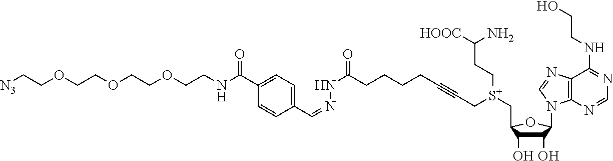 | ETA- AdoHcy- Hydr |
| 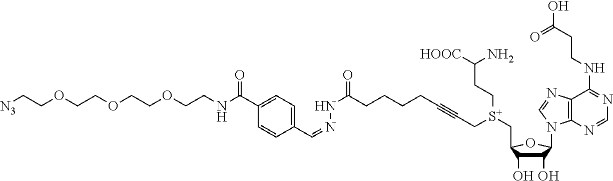 | b-Ala- AdoHcy- Hydr |
| 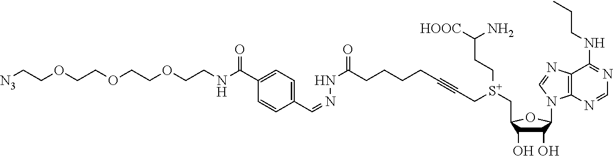 | Propyl- AdoHcy- Hydr |
| 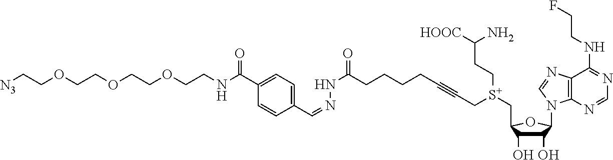 | Fluoro- AdoHcy- Hydr |
| 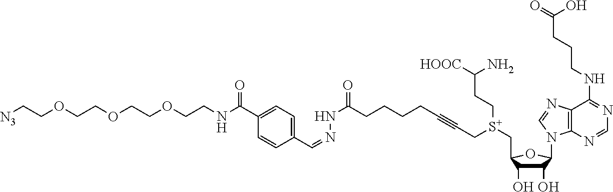 | GABA- AdoHcy- Hydr |
| 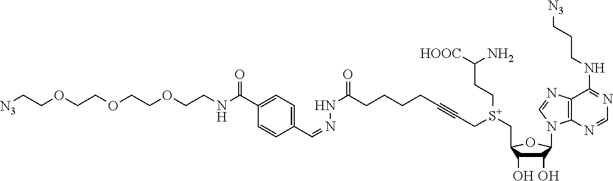 | PAA- AdoHcy- Hydr |
| 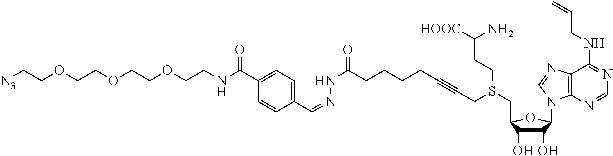 | Allyl- AdoHcy- Hydr |
| 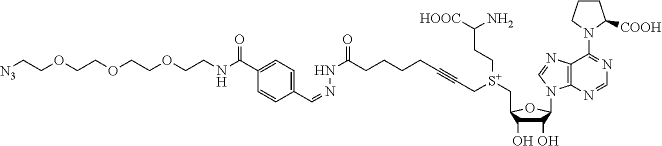 | Pro- AdoHcy- Hydr |

In some embodiments, the compound has the structure:

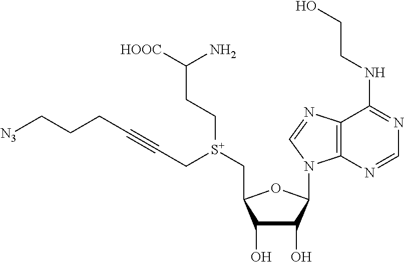

In some embodiments, the compound has the structure:

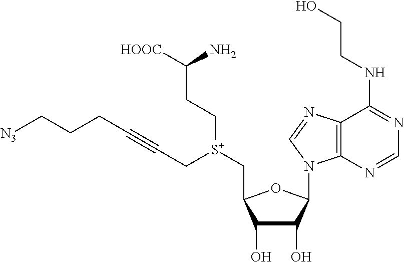

In some embodiments, the compound has the structure:

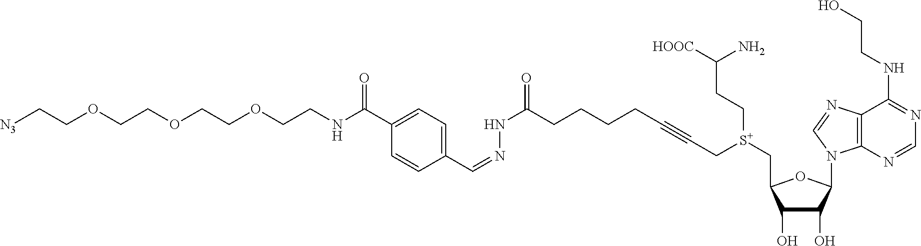

In some embodiments, the compound has the structure:

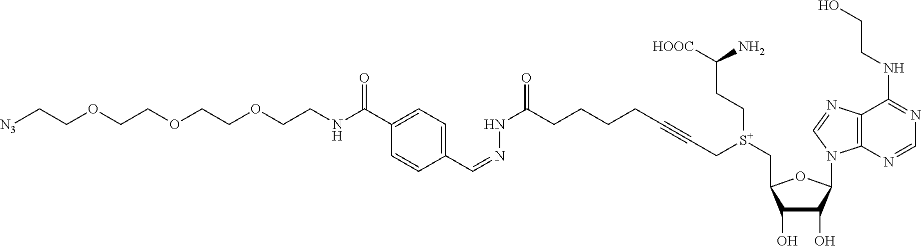

In any embodiment described herein, the compound of formula (I) may be associated with a counter ion. The counter ion may be one or more of a carbonate anion (CO₃²⁻), a hydrogencarbonate (HCO₃⁻), a tetrafluoroborate anion (BF₄⁻), a hexafluorophosphate anion (PF₆⁻), an acetate (OAc⁻), a trifluoroacetate anion, a formate anion, halide (e.g. F⁻, Cl⁻, Br⁻, I⁻), or a sulphonate anion.

The present devisors have surprisingly found that M.Mpel shows increased activity with the AdoMet analogue and ETA-AdoHcy-Hydr compared to their counterparts with no No modification. Without being bound by theory, it is thought that the introduction of the hydroxy group at the Ne position of the adenosine moiety strengthens the binding interaction of the cofactor analogue with M.Mpel. The crystal structure of the ternary DNA-M.Mpel-cofactor complex shows that the cofactor analogue sits in a largely hydrophobic pocket of the enzyme but that there is space to accommodate the Ne modifications (FIG. 1). The crystal structure also suggests that hydrogen bonding between the oxygen of the hydroxy group and the ammonium group of LYS-115 stabilizes the binding of the cofactor analogue to the enzyme. It is thought that other groups which have the potential to form hydrogen bonds with the enzyme, e.g. with LYS-115, will provide similar binding stabilization.

The present disclosure also provides a composition comprising the compound of formula (I). The composition may be a solution, suspension or dispersion of the compound in a suitable solvent, e.g. water or saline. The composition may further comprise one or more reagents selected from: buffers, salts, viscosity modifiers, stabilisers, or pH modifiers. Preferably, the composition is biologically or pharmaceutically acceptable. By this, it will be understood that the components of the composition do not have any detrimental effects on biological molecules which they may come into contact with, in use.

There is also provided a complex of a compound of formula (I) and a methyltransferase.

There is further provided a kit comprising a compound of formula (I), or a composition comprising said compound. The kit may further comprise a methyltransferase.

In some embodiments, the methyltransferase is capable of using S-adenosyl methionine as a cofactor. In other words, the methyltransferase may be an S-adenosyl methionine-(e.g. S-adenosyl-L-methionine-) dependent methyltransferase. In some embodiments the methyltransferase is a DNA methyltransferase. In some embodiments, the methyltransferase is a cytosine-5 methyl transferase. In some embodiments, the methyltransferase is selected from M.Hhal, M.Sssl, M.Mpel, M.Taql, and mutants thereof. M.Mpel can be obtained using the methods described by Wojciechowski et al., Proc Natl Acad Sci USA. 2013 Jan. 2; 110 (1): 105-110. Preparation of M.Sssl is described by Darii et al., Molecular Biology 41, 110-117 (2007). Purification of M. Hhal is described by Kumar et al, Biochemistry (1992), 31 (36), 8648-8653. Preparation of M.Taql is described by Hulz et al., Nucleic Acids Res. 26, 1076-1083 (1998). In some embodiments, the methyltransferase is M.Mpel. In some embodiments, the methyl transferase is a double mutant (Q136A/N₃₇₄A) of M.Mpel. These mutations facilitate the use of AdoMet analogues, such as those described herein, by the enzyme. The skilled person would be capable of engineering further cytosine-5 methyltransferases for site-specific labelling of DNA, using standard molecular biology techniques and the teachings of Lukinavicius et al., Nucleic Acids Research, 40, 22 (2012) pages 11594-11602.

There is also disclosed the use of a compound of formula (I) in a method of modifying a target biomolecule, such as a nucleic acid.

There is further provided a method of modifying a target biomolecule, the method comprising incubating the target biomolecule with a compound of formula (I) and a methyltransferase such that a transferable group (i.e. R₁) of the compound of formula (I) is transferred onto the target biomolecule. The method may be used to form a functionalised biomolecule. The methyltransferase may be one as described herein.

The method may comprise modifying the target biomolecule within cells (e.g. in vitro or ex-vivo), or within a cell lysate. In some embodiments, the cells or cell lysate contain one or more methylases. In some embodiments, the cells or cell lysate contain a plurality of methyltransferases, e.g. at least 2, 3, 4, 5 or 10. It may be that only one or a subset of the methyltransferases are capable of transferring the transferable group from the compound of formula (I) onto the target biomolecule. Alternatively, it may be that one or a subset of the methyltransferases has increased activity with respect to the compound of formula (I), relative to the other methyltransferases present.

The target biomolecule may comprise a nucleic acid, such as DNA, RNA or a mixture thereof. The nucleic acid may be single-stranded or double-stranded. In some embodiments, the target biomolecule is or comprises DNA, e.g. genomic DNA.

It will be appreciated that the incubation will be carried out under conditions which enable the methyltransferase to transfer the transferable group from the compound of formula (I) to the target biomolecule. The skilled person will be capable of determining suitable conditions for a given methyltransferase. In some embodiments, incubation is carried out at a temperature of from 10 to 60° C., from 15 to 50° C., from 20 to 40° C., or from 30 to 37° C. In some embodiments, incubation is carried out for a time sufficient to enable transfer of a transferable group onto all available sites in the target biomolecule. It will be appreciated that the incubation time may depend on factors such as the type of enzyme, the concentration of the target biomolecules, and/or the concentration of the compound of formula (I). For example, incubation may be carried out for a period of time of from 5 minutes to 5 hours, from 10 minutes to 4 hours, from 15 minutes to 3 hours, from 30 minutes to 2 hours, or from 1 hour to 1.5 hours.

In some embodiments, the method may further comprise cleaving the target biomolecule. For example, a DNA or RNA target biomolecule may be cleaved into fragments. Cleavage may be carried out before or after the target biomolecule is incubated with the compound of formula (I) and the methyltransferase. Thus, in some embodiments of the method, the target biomolecule may be a DNA or RNA fragment.

In some embodiments, the transferrable group (i.e. the R₁ moiety of the compound of formula (I)) that is transferred onto the target biomolecule comprises a hydrolysable moiety. In some embodiments, the method further comprises hydrolysing the hydrolysable moiety of the transferrable group.

Additionally, or alternatively, the transferrable group may comprise a detectable label, such as a chromophore, a fluorophore, a radioactive or stable rare isotope.

Additionally, or alternatively, the transferrable group may comprise a functional group that enables further modification of the biomolecule. The method may comprise reacting the functional group with a further reagent, for example to provide a specific functionality. In some embodiments, the reaction between the functional group and the further reagent is a click reaction, such as a strain-promoted alkyne-azide cycloaddition (SPAAC). It may be that the functional group comprises or consists of an azide, while the further reagent comprises an alkyne, such as a sterically-strained (e.g. a ring-strained) alkyne. Alternatively, it may be that the functional group comprises or consists of an alkyne (e.g. a terminal alkyne), while the further reagent comprises an azide.

In some embodiments, the method comprises attaching a label to the functional group, thereby forming a labelled biomolecule. In some embodiments, a labelled biomolecule is formed by reacting the functional group directly with the label, or with a moiety comprising a label. Alternatively, the labelled biomolecule may be formed by first reacting the functional group with a further reagent to form a modified functional group, and then reacting the modified functional group with the label, or a moiety comprising a label. For example, the transferable group may comprise a terminal azide which can be reacted with a sterically-strained alkyne, such as DBCO, which is bound to a detectable label, such as a fluorophore, to form a functionalized biomolecule via a triazole linkage.

The method may further comprise separating modified, functionalised or labelled biomolecules from non-modified, non-functionalised or non-labelled biomolecules.

In some embodiments, the method comprises capturing the modified, functionalised or labelled biomolecules. For example, the transferrable group may comprise an affinity tag which enables capture of a modified or functionalised biomolecule, e.g. using a suitable column. Additionally, or alternatively, it may be that the transferrable group comprises a functional group (i.e. R₅) that is capable of reacting with a label or a ligand to form a labelled biomolecule. The label or ligand may enable capture of the biomolecule. For example, the label may comprise or consist of a biotin moiety or a protein tag (e.g. CLIP-tag, a SNAP-tag, or a maltose binding protein).

In some embodiments, the method further comprises detecting modified or functionalised biomolecules. This may be carried out by, for example, detecting the presence of a detectable label present in the transferable group that has been transferred onto the target biomolecule. For example, fluorescent labelling may be used to visualise the labelling pattern on a DNA or RNA sequence that is introduced by a given methyltransferase.

In some embodiments, the method may further comprise analysing the modified or functionalised biomolecule. Analytical methods may include microscopy, sequencing, fluorimetry, imaging, UV-visible absorption spectroscopy, real-time or quantitative PCR (or other nucleic acid amplification technique), mass spectrometry, chromatography, electrophoresis, and combinations thereof.

As such, the disclosure also provides for the use of AdoMet analogues (i.e. the AdoMet analogue of formula (I) for therapy or diagnosis and/or for use in preparing samples for analysis (e.g. nucleic acid amplification, DNA and RNA sequencing and so on). The disclosure may also provide for the use of AdoMet analogues (i.e. the AdoMet analogue of formula (I) for analysing biomolecules in liquid biopsy samples (e.g. analysing circulating cell-free DNA, RNA or proteins in liquid biopsy samples).

Accordingly, there may be further provided a biomolecule (e.g. DNA or RNA molecule or base or fraction thereof or protein or amino acid or peptide or polypeptide thereof) having bonded thereto a molecule R₁, wherein
R₁ has the structure [R₅]_{q}—[L₁]ₚ—[HM]ₙ—[L₂]ₘ—U—CH₂—
L₁ is a bond or a linker;
HM is a hydrolysable moiety;
L₂ is a linker;
U comprises an unsaturated group selected from an alkene, an alkyne, an aromatic group (e.g. aryl), a carbonyl group, and a sulphur atom comprising one or two S═O bonds; m, n, p and q are each independently selected from 0 and 1; and
R₅ comprises a heavy atom or a heavy atom cluster suitable for phasing of X-ray diffraction data, a radioactive or stable rare isotope, a fluorophore, a fluorescence quencher, an affinity tag, a crosslinking agent, a nucleic acid cleaving reagent, a spin label, a chromophore, a protein, peptide or amino acid which may optionally be modified a nucleotide, nucleoside or nucleic acid which may optionally be modified, a carbohydrate, a lipid, a transfection reagent, an intercalating agent, a nanoparticle or bead, or a functional group,
wherein the functional group is selected from the group consisting of: an amino group (including a protected amino), a thiol group, a 1,2-diol group, a hydrazino group, a hydroxyamino group, a haloacetamide group, a maleimide group, a cyanide group, a cyclic hydrocarbon (such as a bridged cyclic hydrocarbon (e.g. norbornene) or a cycloalkyl group (e.g. a C₃₋₆ cycloalkyl), a halo group (e.g. —F, —Cl, —Br, —I), an aldehyde group, a ketone group, a 1,2-aminothiol group, a azido group, an isothiocyanate or thiocyanate group, an alkene group, such as a terminal alkene, an alkyne group, such as a terminal alkyne group, a 1,3-diene function, a dienophilic function (e.g. an activated carbon-carbon double bond), an arylhalide group, an arylboronic acid group, a terminal haloalkyne group, a terminal silylalkyne group, —N═C═O; —N═C═S, —O—C(O)NH₂, a protected amino, a group comprising a sterically strained alkyne or alkene (such as norbornene or DBCO), a nitrone, a tetrazine, a tetrazole, and 1,2-aminothiol group.

The biomolecule may be isolated. Attachment of the R₁ chain may allow for isolation and/or enrichment of the biomolecule. Accordingly, there is further disclosed an enriched sample of a biomolecule (e.g. DNA or RNA molecule or base or fraction thereof or protein or amino acid or peptide or polypeptide thereof) having an R₁ chain attached thereto.

There may be further provided a catalytically active complex of an AdoMet analogue of formula (I), a methyltransferase and a biomolecule (e.g. DNA or RNA molecule or base or fraction thereof or protein or amino acid or peptide or polypeptide thereof).

There is also disclosed the use of the AdoMet analogues of formula (I) in binding a biomolecule to a solid-phase support. Binding may occur covalently or non-covalently.

There is also disclosed a method for detecting sequence-specific methylation of a target biomolecule, the method comprising:
a) incubating the target biomolecule with a compound of formula (I) and a methyltransferase; and
b) detecting whether a transferable group (i.e. R₁) of the compound of formula (I) has been transferred onto a recognition site of the target biomolecule.

In some embodiments, modification of the recognition site by the transferable group is indicative of the absence of methylation at the recognition site. The present invention thus enables the methylation status of genomic DNA to be determined. This facilitates the detection of diseases associated with an altered methylation status.

The term “recognition site” will be understood as referring to a particular structure or sequence within the target biomolecule that is recognized by the methyltransferase. In embodiments wherein the target biomolecule is DNA or RNA, the recognition site may be a sequence of from 2 to 20, from 3 to 15, from 4 to 12 or from 5 to 10 nucleotides or nucleotide pairs.

In an example, methods of the disclosure may be for analysing DNA, e.g. for epigenetic profiling. The method may comprise:
forming labelled DNA fragments by:
   (a) cleaving or fragmenting genomic DNA into DNA fragments;
   (b) incubating the DNA with a DNA methyltransferase and a compound of formula (I) such that non-methylated CpG sites present in the DNA are selectively functionalized with a transferable group (i.e. R₁) of compound (I), wherein the transferable group comprises a hydrolysable moiety; and
   (c) attaching a label to the transferable moiety (e.g. via a functional group R₅);
separating labelled DNA fragments from non-labelled DNA fragments;
hydrolysing the hydrolysable moiety of the transferrable group on the labelled DNA fragments, so as to release the DNA fragments from the label; and
sequencing the released DNA fragments.

It will be understood that steps (a), (b) and (c) above may be carried out in any order. For example, the label may be attached to the linker before the DNA is functionalized with the linker. The DNA may be functionalized with the linker (to which the label may or may not be already attached) prior to cleaving the DNA, or after cleaving the DNA. Thus, it will be appreciated that step (b) may be carried out on genomic DNA or on DNA fragments.

The present disclosure also provides a method of preparing the compound of formula (I), the method comprising the steps of:
(a) reacting a compound of formula (II)

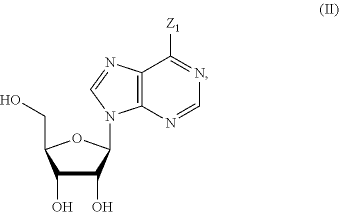

wherein Z₁ is F, Cl, I or Br,
with a halogen donor to form a compound of formula (III);

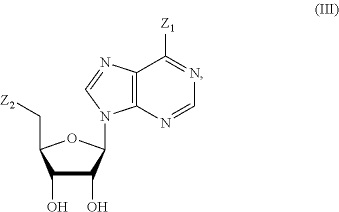

wherein Z₁ and Z₂ are independently selected from F, I, Br and Cl;
(b) reacting the compound of formula (III) with NHR₂R₃, wherein R₂ and R₃ are as defined above, to form a compound of formula (IV);

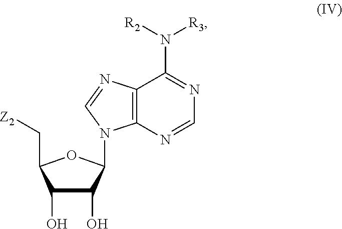

wherein Z₂ is as defined above,
(c) reacting the compound of formula (IV) with homocysteine (e.g. L-homocysteine) or selenohomocysteine (e.g. L-selenohomocysteine) to form a compound of formula V;

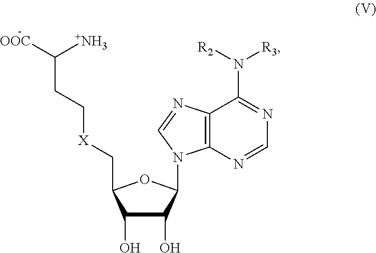

wherein X is Se or S;
and
(d) reacting the compound of formula (V) with R₁-LG to form the compound of formula (I), wherein LG is a leaving group. In some embodiments, the leaving group is selected from halo (e.g. F, Cl, Br or I) or sulfonyl (e.g. tosyl, brosyl, nosyl, mesyl, triflyl, tresyl).

In some embodiments, the halogen donor is selected from: I₂, Br₂, Cl₂; thionyl chloride; or chloro-diisopropylamine. In some embodiments, the halogen donor is I₂.

Optionally, step (a) is carried out in the presence of a base e.g. imidazole, pyridine, or N,N,N,N,N,N-hexamethylphosphoric triamide.

In some embodiments, such as when the halogen donor is |2, step (a) is carried out in the presence of triphenylphosphine (PPh₃), or derivatives thereof, or 5,10,15,20-tetraphenyl-21H, 23H-porphine.

In some embodiments, step (a) is carried out in the presence of a solvent, e.g. polar aprotic solvent, such as acetonitrile or N-methyl-2-pyrrolidone (NMP). Some bases e.g. N,N,N,N,N,N-hexamethylphosphoric triamide can also function as solvents.

In some embodiments, in step (a) the compound of formula (II) is reacted with 12 in the presence of PPh₃ (or derivatives thereof) and a base, optionally wherein the base is imidazole.

In some embodiments, step (b) is carried out in the presence of a (further) base, optionally wherein the base is pyridine or NEt₃. In some embodiments, the NHR₂R₃ reagent itself is used as the base.

In some embodiments, step (c) comprises reacting the compound of formula (IV) with L-homocysteine.

Alternatively, step (c) may comprise reacting the compound of formula (IV) with a mixture of L-homocysteine and D-homocysteine. In this embodiment the method may comprise an additional step of separating the resulting isomers. The isomers may be separated after step (c) and before step (d). Alternatively, separation of the desired isomer may be carried out after step (d).

In some embodiments, step (d) is carried out in the presence of a silver salt. Suitable silver salts include AgClO₄, AgNO₃ and CF₃SO₂OAg.

In some embodiments, step (d) is carried out in the presence of an acid. In some embodiments, the acid is an organic acid. Suitable acids include formic acid, ethanoic acid and mixtures thereof.

The present disclosure also provides an intermediate compound of Formula (III)

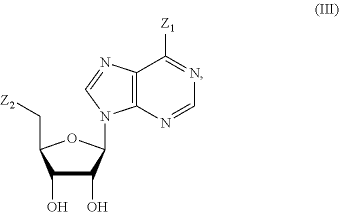

wherein Z₁ and Z₂ are independently selected from I, Br, F and Cl.

In some embodiments, Z₁ is Cl. In some embodiments, Z₂ is I. In some embodiments Z₁ is Cl and Z₂ is I. Thus, in some embodiments the intermediate of formula (III) is:

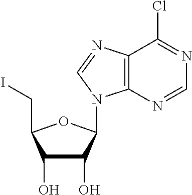

There is also provided an intermediate of formula (IV)

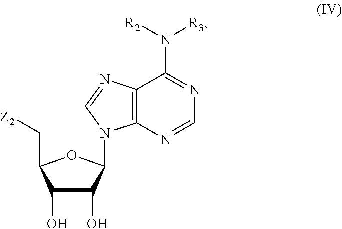

wherein R₂, R₃ and Z₂ are as defined above.

In some embodiments, Z₂ is I.

The cofactors of the disclosure can be used in methods and assays for modifying, labelling and/or analyzing nucleic acids, including but not limited to fluorescent DNA labelling, targeted enrichment of genomic DNA, epigenetic analysis, structural variant analysis and optical mapping.

### EXAMPLES

The disclosure is further illustrated by the following non limiting examples.

### Example 1: General Synthesis

Scheme 1 below is a reaction scheme for the synthesis of Ne-substitued AdoHyc/AdoMet analogues as described herein, according to an embodiment of the present disclosure.

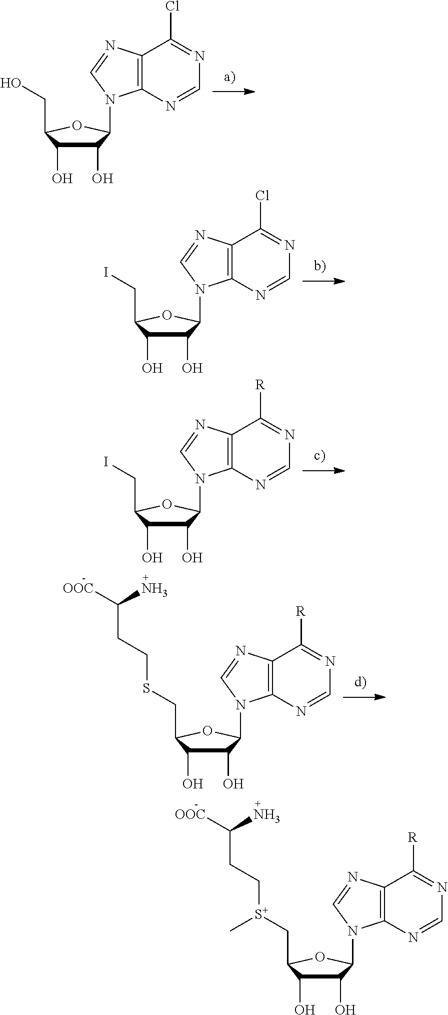

In some embodiments, the reaction conditions are as follows: (a) 12, PPh₃, imidazole, NMP, 24 h, 76%; (b) linker, NEts, water/MeOH (yield 46-95%); (c) L-homocysteine, 1M NaOH, MeOH 100° C. (yield 35-98%); (d) CH₃l, AgCIO4, HCOOH/CH₃COOH (1:1), 30° C. (yield 35-69%).

### Example 2: Synthesis of Cofactor Analogues

### Synthesis of (2R,3R,4S,5S)-2-(6-chloro-9H-purin-9-yl)-5-(chloromethyl)tetrahydrofuran-3,4-diol (5′,6-diCl-Ade)

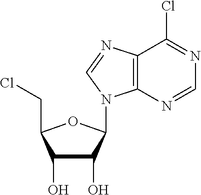

To a cold suspension of 6-chloropurine riboside (1 g, 3.5 mmol) in acetonitrile (10 mL), distilled thionyl chloride (0.76 mL, 3 eq) was added. Then pyridine (0.56 mL, 2 eq) was added and the reaction was stirred at 0° C. for 4 hours and overnight at room temperature. Then, the solvent was removed under reduces pressure and the sample was dissolved in 20 mL of MeOH. 1 ml of water and 2 mL of 35% aqueous solution of ammonia was added. The reaction was stirred for 3 hours. After 1 hour additional 0.6 mL of ammonia was added. The solvent was removed under reduced pressure, 25 mL of 5% citric acid was added and product was extracted with ethyl acetate. The organic layer was washed with NaHCO₃, brine and dried over anhydrous Na₂SO₄. The solvent was removed under reduced pressure yielding a yellow solid (853.4 mg, 80%): 1H NMR (400 MHZ, DMSO-d6) δ 8.92 (s, 1H, 8-H), 8.83 (s, 1H, 2-H), 6.08 (d, J=5.3 Hz, 1H, 1′-H), 5.72 (br. s, 1H, 2′-OH), 5.56 (br. s, 1H, 3′-OH), 4.80-4.73 (m, 1H, 2′-H), 4.31-4.23 (m, 1H, 3′-H), 4.16 (ddd, J=6.3, 4.9, 4.2 Hz, 1H, 4′-H), 3.97 (dd, J=11.7, 4.9 Hz, 1H, 5′-H), 3.88 (dd, J=11.7, 6.3 Hz, 1H, 5″-H); 13C NMR (101 MHZ, DMSO) õ 151.92 (6-C), 151.65 (2-C), 149.48 (4-C), 146.10 (8-C), 131.44 (5-C), 88.17 (1′—C), 83.98 (4′—C), 72.93 (2′—C), 71.14 (3′—C), 44.71 (5′—C); TOF MS ES (−) m/z [M+C₁]-calcd: 338.9818 found: 338.9815.

### Synthesis of (2R,3R,4S,5S)-2-(6-chloro-9H-purin-9-yl)-5-(iodomethyl)tetrahydrofuran-3,4-diol (5′-1,6-Cl-Ade)

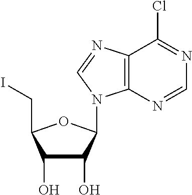

To a cold solution of 6-chloropurine riboside (500 mg, 1.75 mmol) in NMP (3 mL), imidazole (773.5 mg, 6.5 eq) and triphenylphosphine (1516 mg, 3.3 eq) were added. Then solution of iodine (1464 mg, 3.3 eq.) in 2 mL of NMP was added dropwise and the reaction was stirred overnight at room temperature. Then, 10 ml of water was added and product was extracted with ethyl acetate. The organic layer was washed with brine and dried over anhydrous Na₂SO₄. The solvent was removed under reduced pressure, the resulting yellow oil was stored overnight in the fridge. Crystallized triphenylphosphine oxide was filtered off and the crude was purified by preparative RP-HPLC (50-100% MeOH in water over 60 minutes). Collected fraction was lyophilized yielding a white solid (526.8 mg, 76%): 1H NMR (400 MHZ, DMSO-d6) δ 8.94 (s, 1H, 8-H), 8.84 (s, 1H, 2-H), 6.07 (d, J=5.5 Hz, 1H, 1′-H), 5.69 (d, J=5.7 Hz, 1H, 2′-OH), 5.55 (d, J=5.2 Hz, 1H, 3′-OH), 4.82 (ddd, J=5.7, 5.1 Hz, 1H, 2′-H), 4.21 (ddd, J=5.2, 3.8 Hz, 1H, 3′-H), 4.04 (ddd, J=7.0, 5.8, 3.8 Hz, 1H, 4′-H), 3.62 (dd, J=10.5, 5.8 Hz, 1H, 5′-H), 3.49 (dd, J=10.5, 7.0 Hz, 1H, 5″-H); 13C NMR (101 MHZ, DMSO) δ 151.64 (2-C), 145.99 (8-C), 87.91 (1′—C), 83.92 (4′—C), 72.84 (2′—C), 72.75 (3′—C), 7.23 (5′—C). TOF MS ES (+) [M+H]⁺ calcd: 396.9559, found: 396.9561

### Synthesis of 4-((9-((2R,3R,4S,5S)-5-(chloromethyl)-3,4-dihydroxytetrahydrofuran-2-yl)-9H-purin-6-yl) amino) butanoic acid (5′—Cl, 6-GABA-Ade)

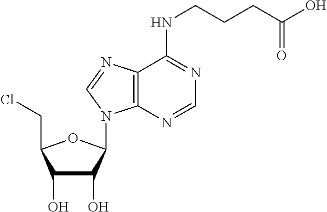

To a solution of 5′,6-diCl-Ade (300 mg, 0.99 mmol) in MeOH (3 mL), GABA (305.91 mg, 3 eq) in 1 ml of water was added. Then triethylamine (821 μL, 6 eq.) was added and the reaction was stirred for 8 hours. The MeOH was removed under reduced pressure and pH was adjusted to 3 with 1M HCl. The precipitate was collected and washed with cold water and dried over P₂O₅ yielding an off-white solid and used in the next step with no further purification (264.5 mg, 72%): 1H NMR (400 MHZ, DMSO-d6) δ 12.03 (s, 1H, COOH), 8.34 (s, 1H, 2-H), 8.22 (s, 1H, 8-H), 7.91 (s, 1H, NH), 5.94 (d, J=5.6 Hz, 1H, 1′-H), 5.60 (s, 1H, 2′-OH), 5.46 (s, 1H, 3′-OH), 4.76 (dd, J=5.9, 4.8 Hz, 1H, 2′-H), 4.23 (dd, J=4.8, 3.9 Hz, 1H, 3′-H), 4.09 (ddd, J=6.4, 5.1, 3.9 Hz, 1H, 4′-H), 3.95 (dd, J=11.6, 5.1 Hz, 1H, 5′-H), 3.84 (dd, J=11.6, 6.4 Hz, 1H, 5″-H), 3.49 (s, 2H, NHCH₂CH₂), 2.27 (t, J=7.4 Hz, 2H, CH₂CH₂COOH), 1.82 (tt, J=7.4, 7.2 Hz, 2H, CH₂CH₂CH₂); 13C NMR (101 MHz, DMSO) δ 152.40 (8-C), 139.29 (2-C), 87.22 (1′—C), 83.42 (4′—C), 72.40 (2′—C), 71.01 (3′—C), 44.56 (5′-C), 38.82 (NHCH₂CH₂), 30.90 (CH₂CH₂COOH), 24.27 (CH₂CH₂CH₂). TOF MS ES (−) m/z [M−H] calcd: 370.0918 found: 370.0916.

### Synthesis of 4-((9-((2R,3R,4S,5S)-5-(iodomethyl)-3,4-dihydroxytetrahydrofuran-2-yl)-9H-purin-6-yl)amino) butanoic acid (5′-1,6-GABA-Ade)

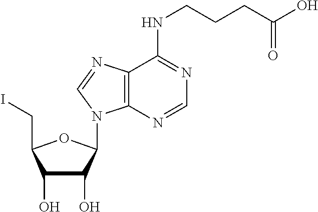

To a solution of 5′-1,6-Cl-Ade (150 mg, 0.38 mmol) in MeOH (1.5 mL), GABA (117 mg, 3 eq) in 200 μL of water was added. Then triethylamine (317 μL, 6 eq.) was added and the reaction was stirred for 8 hours. The MeOH was removed under reduced pressure and pH was adjusted to 4-5 with 1M HCl. The precipitate was collected and washed with cold water and dried over P₂O₅ yielding a white solid and used in the next step with no further purification (129 mg (95% purity with traces of GABA and TEA), 70%): 1H NMR (400 MHZ, DMSO-d6) δ 11.98 (s, 1H, COOH), 8.37 (s, 1H, 2-H), 8.22 (s, 1H, 8-H), 7.91 (s, 1H, NHCH₂), 5.93 (d, J=5.7 Hz, 1H, 1′-H), 5.72-5.33 (m, 2H, 2′-OH, 3′-OH), 4.81 (dd, J=5.7, 5.1 Hz, 1H, 2′-H), 4.20-4.16 (m, 1H, 3′-H), 4.04-3.95 (m, 1H, 4′-H), 3.66-3.57 (m, 1H, 5′-H), 3.56-3.41 (m, 3H, 5″-H, NHCH₂CH₂), 2.28 (t, J=7.3 Hz, 2H, CH₂CH₂COOH), 1.82 (tt, J=7.3, 7.2 Hz, 2H, CH₂CH₂CH₂). 13C NMR (101 MHZ, DMSO-H₆) 6 174.29 (COOH), 152.68 (8-C), 139.72 (2-C), 87.53 (1′—C), 83.91 (4′—C), 73.19 (3′—C), 72.78 (2′—C), 40.15 (5′—C, 5″—C, NHCH₂, under DMSO peak), 31.17 (CH₂CH₂COOH), 24.52 (CH₂CH₂CH₂). TOF MS ES (−) m/z [M−H]⁻ calcd: 462.0274; found: 462.0284.

### Synthesis of 2-((9-((2R,3R,4S,5S)-3,4-dihydroxy-5-(chloromethyl)tetrahydrofuran-2-yl)-9H-purin-6-yl)amino) ethan-1-aminium formate (5′—Cl, 6-EDA-Ade)

### HCOO⁻

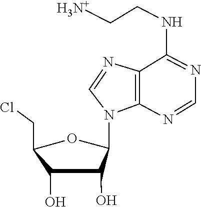

To a solution of 5′,6-diCl-Ade (150 mg, 0.38 mmol) in MeOH (1.5 mL), EDA (152 μL, 6 eq) was added and reaction was stirred for 2 hours. The MeOH was removed under reduced pressure and pH was adjusted to 3 with 1M HCl. Resulting solution was purified by preparative RP-HPLC (3-100% MeOH in 20 mM ammonium formate buffer pH 3.5 over 60 minutes). The collected fraction was lyophilized yielding a white solid as a formic salt (168.8 mg, 95%): 1H NMR (400 MHz, D20) δ 8.41 (s, 1H, HCOO), 8.31 (s, 1H, 2-H), 8.25 (s, 1H, 8-H), 6.06 (d, J=5.3 Hz, 1H, 1′-H), 4.46 (dd, J=5.3, 4.7 Hz, 1H, 2′-H), 4.44-4.38 (m, 1H, 3′-H under solvent peak), 3.95-3.84 (m, 4H, 5′-H, 5″-), 3.33-3.28 (m, 2H, CH₂CH₂NH₃+); 13C NMR (101 MHZ, D2O) δ 170.91, 154.63, 152.75, 148.40, 139.67, 119.11, 87.16, 83.37, 73.44, 70.67, 44.10, 39.27, 38.12; TOF MS ES (+) m/z [M+H]⁺ calcd: 329.1129; found: 329.1136.

### Synthesis of 2-((9-((2R,3R,4S,5S)-3,4-dihydroxy-5-(iodomethyl)tetrahydrofuran-2-yl)-9H-purin-6-yl)amino) ethan-1-aminium formate (5′-1,6-EDA-Ade,)

### HCOO⁻

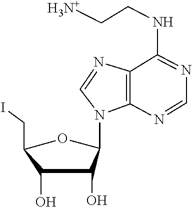

To a solution of 5′-1,6-Cl-Ade (150 mg, 0.38 mmol) in MeOH (1.5 mL), EDA (152 μL, 6 eq) was added and reaction was stirred for 2 hours. The MeOH was removed under reduced pressure and pH was adjusted to 3 with 1M HCl. Resulting solution was purified by preparative RP-HPLC (3-50% MeOH in 20 mM ammonium formate buffer pH 3.5 over 60 min). The collected fraction was lyophilized yielding an amorphic off-white solid as a formic salt (168.8 mg, 95%): 1H NMR (400 MHZ, DMSO-d6) δ 8.43-8.38 (m, 3H, 2-H, 2x HCOO⁻), 8.26 (s, 1H, 8-H), 7.98 (br. s, 1H, NHCH₂CH₂NH₃+), 5.93 (d, J=5.8 Hz, 1H, 1′-H), 4.80 (dd, J=5.4 Hz, 1H, 2′-H), 4.17 (dd, J=5.1, 3.6 Hz, 1H, 3′-H), 4.02-3.95 (m, 1H, 4′-H), 3.75-3.57 (m, 3H, 5′-H, NHCH₂CH₂NH₃+), 3.46 (dd, J=10.4, 7.0 Hz, 1H, 5″-H), 2.97 (t, J=6.3 Hz, 2H, NHCH₂CH₂NH₃+); 13C NMR (101 MHZ, DMSO) δ 165.59, 152.56, 140.01, 129.38, 83.95, 79.20, 73.21, 72.88, 28.06, 7.97; TOF MS ES (+) m/z [M+H]⁺ calcd: 421.0485; found: 421.0496.

### Synthesis of 3-((9-((2R,3R,4S,5S)-5-(chloromethyl)-3,4-dihydroxytetrahydrofuran-2-yl)-9H-purin-6-yl)amino) propanoic acid (5′—Cl, 6-β-Ala-Ade)

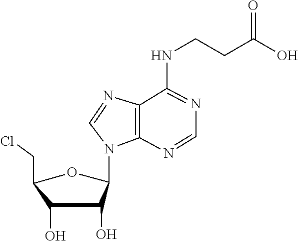

To a solution of 5′,6-diCl-Ade (300 mg, 0.987 mmol) in MeOH (3 mL), β-alanine solution (167 mg, 1.9 eq.) in 800 μL of water and triethylamine (520 μL, 3.8 eq.) were added and reaction was stirred for 8 hours. pH was adjusted to 3 with 1M HCl. Resulting off-white precipitate was washed with cold MeOH and water, and used without further purification (329 mg, 93%): 1H NMR (400 MHZ, DMSO-d6) δ 12.22 (br. s, 1H, —COOH), 8.37 (s, 1H, 2-H), 8.27 (s, 1H, 8-H), 7.84 (s, 1H, NHCH₂), 5.95 (d, J=5.6 Hz, 1H, 1′-H), 4.77 (dd, J=5.6, 5.1 Hz, 1H, 2′-H), 4.25 (dd, J=5.1, 3.5 Hz, 1H, 3′-H), 4.11 (ddd, J=6.4, 5.1, 3.5 Hz, 1H, 4′-H), 3.96 (dd, J=11.6, 5.1 Hz, 1H, 5′-H), 3.85 (dd, J=11.6, 6.4 Hz, 1H, 5″-H), 3.78-3.61 (m, 2H, NHCH₂CH₂), 2.60 (t, J=7.2 Hz, 2H, CH₂CH₂COOH); 13C NMR (101 MHZ, DMSO) δ 173.07, 154.43, 152.69 (8-C), 148.69, 139.77 (2-C), 119.63, 87.52 (1′—C), 83.72 (4′—C), 72.70 (2′—C), 71.29 (3′—C), 44.84 (5′—C), 36.07 (NHCH₂CH₂), 33.77 (CH₂CH₂COOH). TOF MS ES (−) m/z [M−H]: calcd: 356.0762; found: 356.0763.

### Synthesis of 3-((9-((2R,3R,4S,5S)-5-(iodomethyl)-3,4-dihydroxytetrahydrofuran-2-yl)-9H-purin-6-yl)amino) propanoic acid (5′-1,6-β-Ala-Ade)

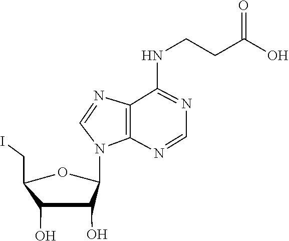

To a solution of 5′,6-diCl-Ade (300 mg, 0.987 mmol) in MeOH (3 mL), β-alanine solution (167 mg, 1.9 eq.) in 800 μL of water and triethylamine (520 μL, 3.8 eq.) were added and reaction was stirred for 8 hours. pH was adjusted to 3 with 1M HCl. Resulting off-white precipitate was washed with cold MeOH and water, and used without further purification (329 mg, 93%); 1H NMR (400 MHZ, DMSO-d6) δ 8.38 (s, 1H, 2-H), 8.25 (s, 1H, 8-H), 7.81 (br. s, 1H, NHCH₂CH₂COOH), 5.93 (d, J=5.7 Hz, 1H, 1′-H), 4.81 (dd, J=5.5 Hz, 1H, 2′-H), 4.18 (dd, J=4.3 Hz, 1H, 3′-H), 4.03-3.95 (m, 1H, 4′-H), 3.68 (br. s, 2H, NHCH₂CH₂COOH), 3.61 (dd, J=10.5, 5.9 Hz, 1H, 5′-H), 3.47 (dd, J=10.5, 6.9 Hz, 1H, 5″-H), 2.59 (t, J=7.2 Hz, 2H, NHCH₂CH₂COOH); 13C NMR (101 MHZ, DMSO) δ 139.89, 83.91, 73.18, 72.79, 54.93, 45.53, 8.98, 7.82. TOF MS ES (−) m/z [M−H] calcd: 448.0118; found: 448.0128.

### Synthesis of (9-((2R,3R,4S,5S)-3,4-dihydroxy-5-(iodomethyl)tetrahydrofuran-2-yl)-9H-purin-6-yl)-L-proline (5′-1,6-Pro-Ade)

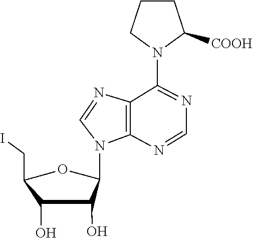

To a solution of 5′-1,6-Cl-Ade (100 mg, 0.253 mmol) in MeOH (1 mL) and water (100 μL), L-Proline*HCl (174.5 mg, 6 eq.) and triethylamine (211 μL, 6 eq.) were added and reaction was stirred for 8 hours. Resulting solution was purified by preparative RP-HPLC (3-100% MeOH in water over 60 min). The collected fraction was lyophilized yielding a white solid as (55 mg, 46%): 1H NMR (400 MHZ, DMSO-d6) δ 12.55 (br. s, 1H, —COOH), 8.45-8.19 (m, 2H, 2-H, 8-H), 6.00-5.92 (m, 1H, 1′-H), 5.61 (d, J=6.0 Hz, 1H, 2′-OH), 5.52-5.44 (m, 1H, 3′-OH), 5.37-5.29 (m, Ha-rot. 1), 4.85-4.76 (m, 1H, 2′-H), 4.69-4.61 (m, Ha-rot. 2), 4.25-4.12 (m, 2H, 3′-OH, Hδ), 4.05-3.95 (m, 1H, 4′-H), 3.84-3.67 (m, 1H, Hδ), 3.66-3.56 (m, 1H, 5′-H), 3.52-3.44 (m, 1H, 5″-H), 2.43-1.72 (m, 3H, HB, Hy, one H under solvent peak). 13C NMR (101 MHZ, DMSO) δ 152.84, 152.50, 140.07, 139.83, 87.85, 84.35, 84.25, 73.65, 73.38, 73.14, 60.92, 60.06, 49.52, 47.94, 31.17, 29.18, 24.89, 22.42, 8.30; TOF MS ES (−) m/z [M−H]-calcd: 474.0274; found: 474.0270.

### Synthesis of (2R,3R,4S,5S)-2-(6-((2-hydroxyethyl)amino)-9H-purin-9-yl)-5-(iodomethyl)tetrahydrofuran-3,4-diol (5′-1,6-ETA-Ade)

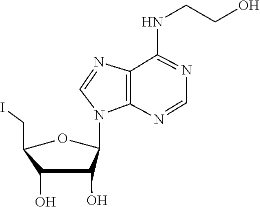

To a solution of 5′-1,6-Cl-Ade (100 mg, 0.253 mmol) in MeOH (1 mL), ethanolamine (91 μL, 6 eq.) and triethylamine (211 μL, 6 eq.) were added and reaction was stirred for 8 hours. Resulting solution was purified by semi-prep HPLC (3-100% MeOH in water over 60 min). The collected fraction was lyophilized yielding a white solid as (80 mg, 75%): 1H NMR (500 MHz, DMSO-d6) δ 8.37 (s, 1H, 2-H), 8.23 (br. s, 1H, 8-H), 7.66 (br. s, 1H, NHCH₂), 5.93 (d, J=5.7 Hz, 1H, 1′-H), 5.57 (d, J=6.0 Hz, 1H, 2′-OH), 5.46 (d, J=5.0 Hz, 1H, 3′-OH), 4.85-4.79 (m, 1H, 2′-H), 4.76 (br. s, 1H, CH₂OH), 4.21-4.15 (m, 1H, 3′-H), 4.02-3.96 (m, 1H, 4′-H), 3.65-3.50 (m, 5H, CH₂CH₂, 5′-H), 3.47 (dd, J=10.4, 6.9 Hz, 1H, 5″-H); 13° C. NMR (126 MHZ, DMSO) δ 154.67, 152.62, 148.71, 139.81, 119.58, 87.53, 83.89, 73.18, 72.77, 59.68, 42.46, 7.83; TOF MS ES (+) m/z [M+H]⁺ calcd: 422.0325; found: 422.0334

### Synthesis of (2R,3R,4S,5S)-2-(6-(allylamino)-9H-purin-9-yl)-5-(iodomethyl)tetrahydrofuran-3,4-diol (5′-1,6-Allyl-Ade)

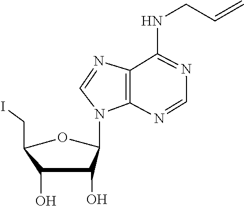

To a solution of 5′-1,6-Cl-Ade (100 mg, 0.1 mmol) in MeOH (1 mL), allylamine (114 μL, 6 eq.) and triethylamine (211 μL, 6 eq.) were added and reaction was stirred for 24 hours. The precipitate was then filtered off and washed with cold water, methanol and dried over P₂O₅ yielding a white solid (66 mg, 63%) which was used in the next step without further purification: 1H NMR (400 MHZ, DMSO-d6) δ 8.39 (s, 1H, 2-H), 8.23 (br. s, 1H, 8-H), 8.04 (br. s, 1H, NHCH₂), 6.02-5.89 (m, 2H, 1′-H, CH₂CH═CH₂), 5.60 (d, J=6.1 Hz, 1H, 2′-OH), 5.48 (d, J=5.1 Hz, 1H, 3′-OH), 5.20-5.11 (m, 1H, CH₂CH═CHH), 5.08-5.02 (m, 1H, CH₂CH═CHH), 4.87-4.79 (m, 1H, 2′-H), 4.22-4.06 (m, 3H, 3′-H, NHCH₂CH═CH₂), 4.00 (ddd, J=7.0, 5.9, 3.5 Hz, 1H, 4′-H), 3.62 (dd, J=10.4, 5.9 Hz, 1H, 5′-H), 3.48 (dd, J=10.4, 7.0 Hz, 1H, 5″-H); 13C NMR (101 MHZ, DMSO) δ 154.89, 153.10, 140.29, 136.08, 119.96, 115.45, 87.95, 84.38, 73.64, 73.21, 42.40, 8.29; TOF MS ES (+) m/z [M+H]⁺ calcd: 418.0376; found: 418.0386

### Synthesis of (2R,3R,4S,5S)-2-(6-((3-azidopropyl)amino)-9H-purin-9-yl)-5-(iodomethyl)tetrahydrofuran-3,4-diol (5′-1,6-PAA-Ade)

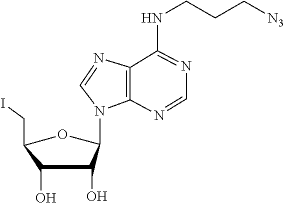

To a solution of 5′-1,6-C₁-Ade (40 mg, 0.1 mmol) in MeOH (0.4 mL), 3-azido-1-propanamine (60.7 mg, 6 eq.) and triethylamine (84 μL, 6 eq.) were added and reaction was stirred for 8 hours. Resulting solution was diluted 10 fold with water/MeOH (1:1), pH adjusted to 7 with 1M HCl and purified by semi-prep HPLC (50-100% MeOH in water over 60 min). The collected fraction was lyophilized yielding a white solid (28.7 mg, 62%): 1H NMR (400 MHZ, Methanol-d₄) δ 8.26 (m, J=2.6 Hz, 2H, 2-H, 8-H), 6.01 (d, J=5.2 Hz, 1H, 1′-H), 4.89-4.82 (m, 1H, 2′-H under solvent peak), 4.30 (dd, J=5.4, 4.1 Hz, 1H, 3′-H), 4.06 (td, J=5.7, 4.1 Hz, 1H, 4′-H), 3.68 (br. s, 2H, NHCH₂CH₂), 3.62 (dd, J=10.7, 5.8 Hz, 1H, 5′-H), 3.50 (dd, J=10.7, 5.8 Hz, 1H, 5″-H), 3.44 (t, J=6.7 Hz, 2H, CH₂CH₂N₃), 1.94 (tt, J=6.7 Hz, 2H, NHCH₂CH₂CH₂N₃); 13C NMR (101 MHZ, MeOD) δ 153.99, 141.00, 90.14, 85.19, 74.92, 74.87, 50.13, 6.26. ESI MS (+) m/z [M+H]⁺ calcd: 461.0541, found: 461.0528

### Synthesis of S-(((2S,3S,4R,5R)-5-(6-((2-carboxyethyl)amino)-9H-purin-9-yl)-3,4-dihydroxytetrahydrofuran-2-yl)methyl)-L-homocysteine (β-Ala-SAH)

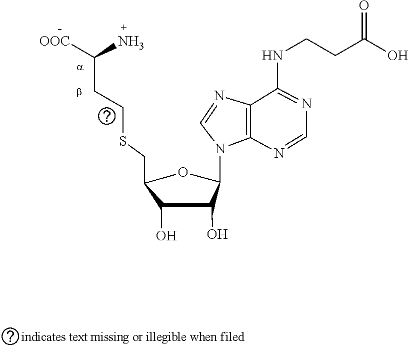

A solution of L-homocysteine (18.5 mg, 2 eq.) in 1M NaOH (200 μL, 3 eq.) was degassed under N₂ for 15 mins followed by addition of a solution of 5′—Cl, 6-B-Ala-Ade (25 mg, 0.07 mmol) or 5′-1,6-B-Ala-Ade (30 mg, 0.067 mmol) in 300 μL of MeOH. The reaction was degassed for additional 10 mins and heated up to 100° C. The progress was monitored by HPLC. The reaction was quenched by addition of 1M HCl to pH 3-4. Reaction was purified by preparative RP-HPLC (3-100% MeOH in 20 mM ammonium formate buffer pH 3.5 over 60 min) and collected fraction was lyophilized to give B-Ala-SAH (12.1 mg, 38%, 24h) and (24.8 mg, 81%, 4.5h) respectively as a white solid. 1H NMR (400 MHZ, DMSO-d6) δ 8.37 (s, 1H, 2-H), 8.25 (s, 1H, 8-H), 7.80 (s, 1H, NHCH₂), 5.90 (d, J=5.6 Hz, 1H, 1′-H), 4.79-4.67 (m, 1H, 2′-H), 4.20-4.12 (m, 1H, 3′-H), 4.09-3.97 (m, 1H, 4′-H), 3.68 (br. s, 2H, NHCH₂CH₂), 3.35-3.29 (m, 1H, Ha), 2.91 (dd, J=13.8, 6.0 Hz, 1H, 5′-H), 2.80 (dd, J=13.8, 6.9 Hz, 1H, 5″-H), 2.66-2.54 (m, 4H, 2Hy, NHCH₂CH₂COOH), 2.05-1.91 (m, 1H, HB), 1.88-1.73 (m, 1H, HB); 13C NMR (101 MHZ, DMSO) δ 173.15, 170.14, 152.64, 139.81, 87.46, 83.68, 72.80, 72.65, 52.95, 33.85, 31.34, 28.10; ESI MS (+) m/z [M+H]⁺ calcd: 457.1500, found: 457.1519

### Synthesis of S-(((2S,3S,4R,5R)-5-(6-((3-carboxypropyl)amino)-9H-purin-9-yl)-3,4-dihydroxytetrahydrofuran-2-yl)methyl)-L-homocysteine (GABA-AdoHcy)

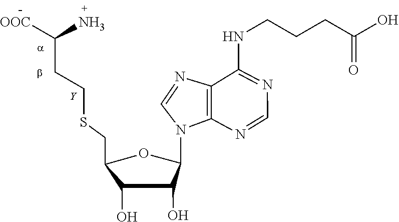

A solution of L-homocysteine (18 mg, 2 eq.) in 1M NaOH (190 μL, 3 eq.) was degassed under N₂ for 15 mins followed by addition of a solution of 5′—Cl, 6-GABA-Ade (25 mg, 0.067 mmol) or 5′-1,6-GABA-Ade (30 mg, 0.065 mmol) in 310 μL of MeOH. The reaction was degassed for additional 10 mins and heated up to 100° C. The progress was monitored by HPLC. The reaction was quenched by addition of 1M HCl to pH 3-4. Reaction was purified by preparative RP-HPLC (3-100% MeOH in 20 mM ammonium formate buffer pH 3.5 over 60 min) and collected fraction was lyophilized to give GABA-AdoHcy (11 mg, 35%, 24h) and (22.6 mg, 74%, 4.5h) respectively as a white solid; 1H NMR (400 MHZ, DMSO-d6) δ 8.36 (s, 1H, 2-H), 8.22 (s, 1H, 8-H), 7.94 (br. s, 1H, NHCH₂), 5.89 (d, J=5.6 Hz, 1H, 1′-H), 4.78-4.67 (m, 1H, 2′-H), 4.18-4.12 (m, 1H, 3;-H), 4.06-3.98 (m, 1H, 4′-H), 3.49 (br. s, 2H, NHCH₂CH₂), 3.36-3.28 (m, 1H, Ha), 2.91 (dd, J=13.8, 5.9 Hz, 1H, 5′-H), 2.81 (dd, J=13.8, 6.9 Hz, 1H, 5″-H), 2.63 (t, J=7.7 Hz, 2H, Hy), 2.26 (t, J=7.4 Hz, 2H, NHCH₂CH₂CH₂COOH), 2.05-1.92 (m, 1H, HB), 1.88-1.75 (m, 3H, HB, NHCH₂CH₂CH₂COOH); 13C NMR (101 MHZ, DMSO) δ 174.61, 170.28, 152.68, 139.58, 87.45, 83.75, 72.85, 72.63, 52.97, 33.85, 31.69, 31.40, 28.16; MS ESI [M+H]⁺ calcd: 471.1662, found: 471.1666

### Synthesis of(S)-2-ammonio-4-((((2S,3S,4R,5R)-5-(6-((2-ammonioethyl)amino)-9H-purin-9-yl)-3,4-dihydroxytetrahydrofuran-2-yl)methyl)thio) butanoate formate (EDA-AdoHcy)

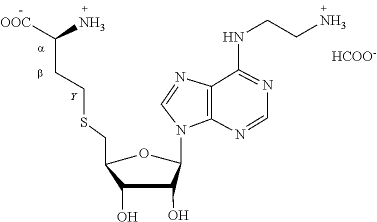

A solution of L-homocysteine (36.2 mg, 2 eq.) in 1M NaOH (403 μL, 3 eq.) was degassed under N₂ for 15 mins followed by addition of a solution of 5′—Cl, 6-EDA-Ade (50 mg, 0.134 mmol) or 5′-1,6-EDA-Ade (62.5 mg, 0.134 mmol). The reaction was degassed for additional 10 mins and heated up to 100° C. The progress was monitored by HPLC. The reaction was quenched by addition of 1M HCl to pH 3-4. Reaction was purified by preparative RP-HPLC (3-50% MeOH in 20 mM ammonium formate buffer pH 3.5 over 60 min) and collected fraction was lyophilized to give EDA-AdoHcy (23.5 mg, 37%, 5h) and (42.5 mg, 67%, 10 min) respectively as a white solid. 1H NMR (400 MHZ, DMSO-d6) δ 8.43-8.39 (m, 3H, 2-H, 2xHCOO-), 8.26 (s, 1H, 8-H), 8.05 (s, 1H, NHCH₂), 5.90 (d, J=5.5 Hz, 1H, 1′-H), 4.71 (dd, J=5.5, 5.0 Hz, 1H, 2′-H), 4.15 (dd, J=5.0, 3.9 Hz, 1H, 3′-H), 4.03 (ddd, J=6.9, 5.7, 3.9 Hz, 1H, 4′-H), 3.68 (br. s, J=8.5 Hz, 2H, NHCH₂CH₂NH₃+), 3.32 (dd, J=6.9, 5.3 Hz, 1H, Ha), 3.00 (t, J=6.2 Hz, 2H, NHCH₂CH₂NH₃+), 2.90 (dd, J=13.9, 5.7 Hz, 1H, 5′-H), 2.81 (dd, J=13.9, 6.9 Hz, 1H, 5″-H), 2.62 (t, J=7.7 Hz, 2H, Hy), 2.03-1.89 (m, 1H, HB), 1.88-1.76 (m, 1H, HB); 13C NMR (101 MHZ, DMSO) δ 165.37, 152.29, 139.65, 87.23, 83.67, 72.66, 72.35, 52.66, 39.11, 38.78, 33.60, 31.21, 27.94. TOF MS ES (+) m/z [M+H]⁺ calcd: 428.1716, found: 428.1724.

### Synthesis of(S)-2-ammonio-4-((((2S,3S,4R,5R)-3,4-dihydroxy-5-(6-((2-hydroxyethyl)amino)-9H-purin-9-yl)tetrahydrofuran-2-yl)methyl)thio) butanoate (ETA-AdoHcy)

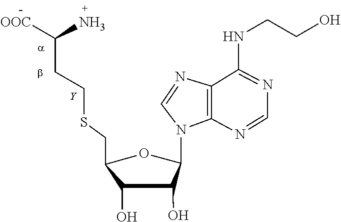

A solution of L-homocysteine (25.7 mg, 2 eq.) in 1M NaOH (210 μL, 2.2 eq.) was degassed under N₂ for 15 mins followed by addition of a solution 5′-1,6-ETA-Ade (40 mg, 0.095 mmol) in 100 μL MeOH. The reaction was degassed for additional 10 mins and heated up to 100° C. The progress was monitored by HPLC. The reaction was quenched by addition of 1M HCl to pH 3-4. Reaction was purified by preparative RP-HPLC (3-100% MeOH in 20 mM ammonium formate buffer pH 3.5 over 60 min) and collected fraction was lyophilized to give (16.8 mg, 41.3%, 4 hours) as a white solid. 1H NMR (400 MHZ, DMSO-d6) δ 8.38 (s, 1H, 2-H), 8.23 (s, 1H, 8-H), 7.75 (br. s, 1H, NHCH₂), 5.90 (d, J=5.7 Hz, 1H, 1′-H), 4.75-4.66 (m, 1H, 2′-H), 4.19-4.10 (m, 1H, 3′-H), 4.09-3.99 (m, 1H, 4′-H), 3.63-3.49 (m, 4H, NHCH₂CH₂OH), 3.47-3.11 (m, 1H, Ha), 2.91 (dd, J=13.8, 6.0 Hz, 1H, 5′-H), 2.81 (dd, J=13.8, 7.0 Hz, 1H, 5″-H), 2.69-2.58 (m, 2H, Hy), 2.08-1.93 (m, 1H, HB), 1.92-1.77 (m, 1H, HB); 13C NMR (101 MHZ, DMSO) δ 170.36, 154.64, 152.68, 148.71, 139.67, 119.50, 87.41, 83.72, 72.91, 72.64, 59.68, 53.02, 42.66, 33.91, 31.42, 28.12; TOF MS ES (+) m/z [M+H]⁺ calcd: 429.1556, found: 429.1564.

### Synthesis of(S)-2-ammonio-4-((((2S,3S,4R,5R)-5-(6-((S)-2-carboxypyrrolidin-1-yl)-9H-purin-9-yl)-3,4-dihydroxytetrahydrofuran-2-yl)methyl)thio) butanoate (Pro-AdoHcy)

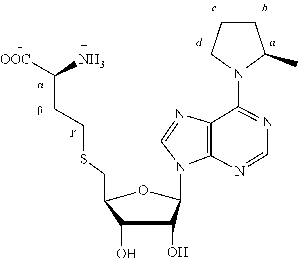

A solution of L-homocysteine (17.1 mg, 2 eq.) in 1M NaOH (190 μL, 2 eq.) was degassed under N₂ for 30 mins followed by addition of 5′-1,6-Pro-Ade (30 mg, 0.063 mmol.) in 110 μL of MeOH. The reaction was degassed for additional 10 mins and heated up to 100° C. The progress was monitored by HPLC. The reaction was quenched by addition of 1M HCl to pH 3-4. Reaction was purified by preparative RP-HPLC (3-100% MeOH in 20 mM ammonium formate buffer pH 3.5 over 60 min) and collected fraction was lyophilized yielding to give (29.9 mg, 98.2%, 3 hours) as a white solid; 1H NMR (400 MHZ, D2O+0.1% TFA) Õ 8.32-8.07 (m, 2H, 2-H, 8-H), 6.04 (d, J=4.8 Hz, 1H, 1′-H), 5.16 (dd, J=8.9, 2.9 Hz, 1H, Ha rot. 1), 4.79 (s, 3H, 2′-H, two proton under solvent peak), 4.56 (dd, J=8.3, 3.4 Hz, 1H, Ha rot. 2), 4.44-4.34 (m, 1H, 3′-H), 4.34-4.26 (m, 1H, 4′-H), 4.25-4.00 (m, 1H), 3.87-3.76 (m, 1H), 3.74-3.61 (m, 1H), 3.08-2.88 (m, 2H, 5′-H, 5″-H), 2.72-2.57 (m, 2H), 2.48-1.85 (m, 4H); 13C NMR (101 MHZ, D2O+0.1% TFA) δ 179.48, 174.01, 170.41, 151.45, 150.85, 149.86, 148.54, 139.20, 138.77, 119.86, 87.44, 83.34, 73.45, 73.25, 72.26, 64.27, 63.10, 53.67, 50.08, 48.90, 33.37, 31.29, 30.30, 29.65, 27.82, 24.33, 22.39. TOF MS ES (+) m/z [M+H]⁺ calcd: 483.1662, found: 483.1675

### Synthesis of(S)-4-((((2S,3S,4R,5R)-5-(6-(allylamino)-9H-purin-9-yl)-3,4-dihydroxytetrahydrofuran-2-yl)methyl)thio)-2-ammoniobutanoate (Allyl-AdoHcy)

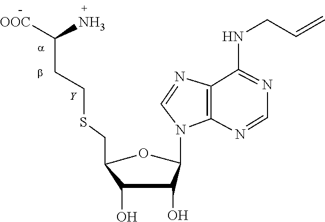

A solution of L-homocysteine (15.8 mg, 2 eq.) in 2M NaOH (84.6 μL, 2.9 eq.) was degassed under N₂ for 15 mins followed by addition of a solution 5′-1,6-Allyl-Ade (25 mg, 0.060 mmol) in 60 μL DMF. The reaction was degassed for additional 10 mins and heated up to 100° C. The progress was monitored by HPLC. The reaction was quenched by addition of 1M HCl to pH 3-4. Reaction was purified by preparative RP-HPLC (3-100% MeOH in 20 mM ammonium formate buffer pH 3.5 over 60 min) and collected fraction was lyophilized to give (24.7 mg, 97%, 1h 30 min) as a white solid. 1H NMR (400 MHZ, DMSO-d6) δ 8.38 (s, 1H, 2-H), 8.22 (s, 1H, 8-H), 8.03 (br. s, 1H, NHCH₂CH═CH₂), 6.01-5.87 (m, 2H, 1′-H, NHCH₂CH═CH₂), 5.19-5.10 (m, 1H, NHCH₂CH═CHH), 5.08-5.02 (m, 1H, NHCH₂CH═CHH), 4.74 (dd, J=5.5 Hz, 1H, 2′-H), 4.19-4.06 (m, 3H, 3′-H, NHCH₂CH═CH₂), 4.05-3.97 (m, 1H, 4′-H), 3.31 (dd, J=7.1, 5.2 Hz, 1H, Ha), 2.92 (dd, J=13.7, 6.2 Hz, 1H, 5′-H), 2.80 (dd, J=13.7, 6.9 Hz, 1H, 5″-H), 2.63 (t, J=7.8 Hz, 2H, HY), 2.06-1.76 (m, 2H, 2xHB); 13C NMR (101 MHZ, DMSO) δ 170.03, 163.92, 152.64, 139.77, 135.70, 115.00, 87.39, 83.60, 72.76, 72.64, 52.99, 33.87, 31.37, 28.09; TOF MS ES (+) m/z [M+H]⁺ calcd: 425.1607, found: 425.1618.

### Synthesis of(S)-2-ammonio-4-((((2S,3S,4R,5R)-5-(6-((3-azidopropyl)amino)-9H-purin-9-yl)-3,4-dihydroxytetrahydrofuran-2-yl)methyl)thio) butanoate (PAA-AdoHcy)

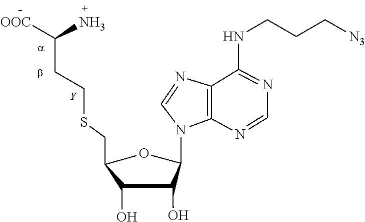

A solution of L-homocysteine (58.8 mg, 4 eq.) in 1M NaOH (430 μL, 4 eq.) was degassed under N₂ for 30 mins followed by addition of 5′-1,6-PAA-Ade (50 mg, 0.108 mmol) in 500 UL of MeOH. The reaction was degassed for additional 10 mins and heated up to 100° C. The progress was monitored by HPLC. The reaction was quenched by addition of 1M HCl to pH 3-4. Reaction was purified by preparative RP-HPLC (3-100% MeOH in 20 mM ammonium formate buffer pH 3.5 over 60 min) and collected fraction was lyophilized yielding PAA-AdoHcy (32.9 mg, 65%, 5 hours) as a white solid; 1H NMR (400 MHZ, DMSO-d6) δ 8.37 (s, 1H, 2-H), 8.23 (s, 1H, 8-H), 7.96 (br. s, 1H, NHCH₂), 5.89 (d, J=5.8 Hz, 1H, 1′-H), 4.72 (dd, J=5.8, 5.1 Hz, 1H, 2′-H), 4.14 (dd, J=5.1, 3.6 Hz, 1H, 3′-H), 4.05-3.99 (m, 1H, 4′-H), 3.54 (br. s, 2H, NHCH₂CH₂), 3.42 (t, J=6.7 Hz, 2H, NHCH₂CH₂CH₂N₃), 3.30 (dd, J=7.1, 5.2 Hz, 1H, Ha), 2.91 (dd, J=13.8, 6.2 Hz, 1H, 5′-H), 2.79 (dd, J=13.8, 6.9 Hz, 1H, 5″-H), 2.62 (t, J=7.8 Hz, 2H, Hy), 2.05-1.93 (m, 1H, HB), 1.89-1.78 (m, 3H, HB, NHCH₂CH₂CH₂N₃); 13C NMR (101 MHZ, DMSO) δ 170.20, 165.64, 154.62, 152.71, 139.73, 87.42, 83.66, 72.85, 72.66, 53.09, 48.59, 37.21, 33.92, 31.46, 28.44, 28.13; TOF MS ES (+) m/z [M+H]⁺ calcd: 468.1778, found: 468.1782

Synthesis of (3-amino-3-carboxypropyl) (((2S,3S,4R,5R)-5-(6-((2-ammonioethyl)amino)-9H-purin-9-yl)-3,4-dihydroxytetrahydrofuran-2-yl)methyl) (8-(2-((Z)-4-((2-(2-(2-(2-azidoethoxy) ethoxy) ethoxy)ethyl) carbamoyl)benzylidene) hydrazineyl)-8-oxooct-2-yn-1-yl) sulfonium (EDA-AdoHcy-Hydr) and (3-amino-3-carboxypropyl) (8-(2-((Z)-4-((2-(2-(2-(2-azidoethoxy) ethoxy) ethoxy)ethyl) carbamoyl)benzylidene) hydrazineyl)-8-oxooct-2-yn-1-yl) (((2S,3S,4R,5R)-3,4-dihydroxy-5-(6-((2-hydroxyethyl)amino)-9H-purin-9-yl)tetrahydrofuran-2-yl)methyl) sulfonium (ETA-AdoHcy-Hydr).

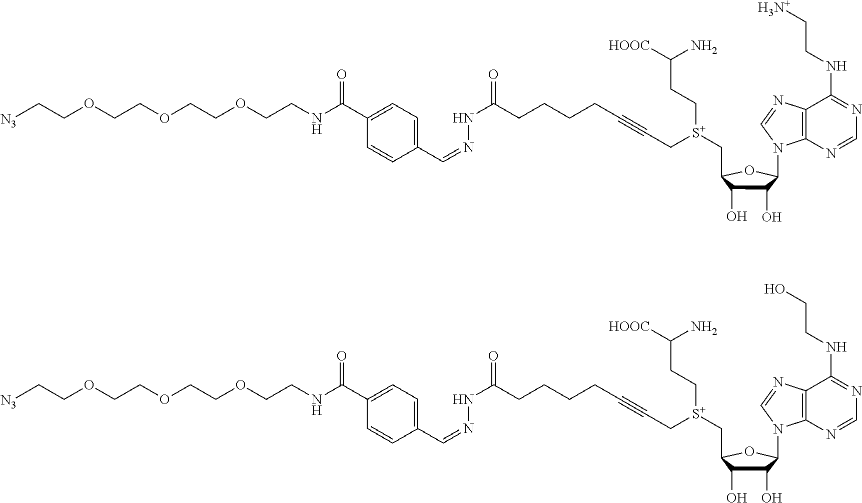

EDA-AdoHcy-Hydr and ETA-AdoHcy-Hydr were synthesized as described in Wilkinson A. A. et al., ACS Cent. Sci., 2020, using ETA-AdoHcy (TOF MS ES (+) m/z [M+H]⁺ calcd: 429.1556, found: 429.1564) and EDA-AdoHcy (TOF MS ES (+) m/z [M+H]⁺ calcd: 428.1716, found: 428.1724) analogues instead of AdoHcy.

### Synthesis of AdoHcy-6-azide cofactor analogues

To a solution of respective N₆-modified AdoHcy analogue in HCOOH/CH₃COOH (1:1), 6-azidohex-2-yn-1-p-toluenesulfonate (various eq) was added slowly at 0° C. The reaction mixture was stirred overnight at 28° C. The resulting mixture was diluted 5 times with water and an excess of linker was extracted with diethyl ether. Then, traces of organic solvent were removed under reduced pressure. The solution was purified by preparative RP-HPLC (3-43.5% MeOH in 20 mM ammonium formate buffer pH 3.5 over 45 min then 43.5-100% over 15 min) and the collected fraction was lyophilized. The product was stored at −20° C. in 0.1% formic acid solution.

### ETA-AdoHcy-6-azide

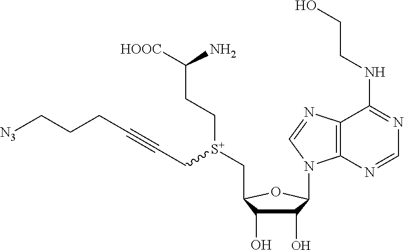

Isomer 1 TOF MS ES (+) m/z M⁺ calcd: 550.2196, found: 550.2211

Isomer 2 TOF MS ES (+) m/z M⁺ calcd: 550.2196, found: 550.2202

### Propyl-AdoHcy-6-azide

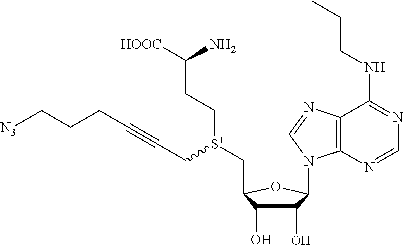

TOF MS ES (+) m/z M⁺ calcd: 548.2404, found: 548.2417

### EDA-AdoHcy-6-azide

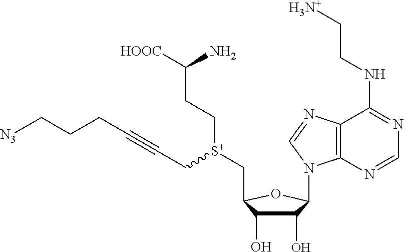

Isomer 1 TOF MS ES (+) m/z M⁺ calcd: 549.2356, found: 549.2341 Isomer 2 TOF MS ES (+) m/z M⁺ calcd: 549.2356, found: 549.2367

Fluoro-AdoHcy-6-azide:

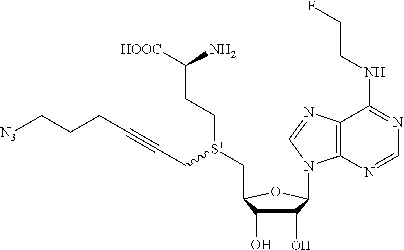

TOF MS ES (+) m/z M⁺ calcd: 552.2153, found: 552.2162

### B-Ala-AdoHcy-6-azide

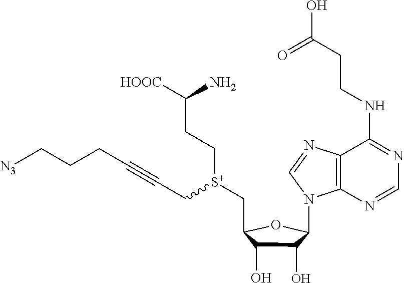

Isomer 1 TOF MS ES (+) m/z M⁺ calcd: 578.2145, found: 578.2151

Isomer 2 TOF MS ES (+) m/z M⁺ calcd: 578.2145, found: 578.2154

### Example 3: Restriction assay using M.Mpel (Q136A, N₃₇₄A), pUC19 and AdoHcy-6-azide analogues

### Methods

For pUC19 restriction assay with M.Mpel and AdoHcy-6-azide analogue, tubes containing 1000 ng pUC19, 138 μg/mL M.Mpel (Q136A, N₃₇₄A) in NEB Cutsmart buffer pH=8.5 in total volume of 10 μl were prepared, apart from first where 20 μl was added. To each tube, AdoHcy-6-azide analogue solution in 0.1% formic acid was added to the first tube to reach desired concentration, then 10 μl was transferred to the next tube and dilution was continued to reach the lowest desired concentration of cofactor. AdoMet solution was added to two control samples to match the highest concentration of AdoHcy-6-azide analogues used in the assay. Three additional controls (10 μL each) were prepared with the same concentrations except the corresponding components were omitted: control of AdoHcy-6-azide analogue at highest concentration with M.Mpel omitted and with both M.Mpel and AdoHcy-6-azide analogue omitted. All the samples were incubated for 1 hour at 37° C. Then, 2 μL of proteinase K solution (20 mg/mL) was added to each sample and then samples were incubated for 1 hour at 50° C. The samples were purified using Zymo DNA Clean-up and concentration columns following manufacturer's protocol with elution into 20 μL of water heated up to 50° C. To 20 μL of each sample 2 μL of Tango buffer (10x) (Thermo Fisher) and 0.3 μL of Hpall (10 U/μL) (Thermo Fisher) were added and samples were incubated for 1 hour at 37° C. Next, 0.3 L of proteinase K (20 mg/mL) was added to all samples and samples were incubated for 1 hour at 50° C. Samples were run on 1% agarose gel (120V for 40 minutes). The method was repeated with further AdoHcy analogues.

### Results

FIG. 2 shows the result of a protection assay of AdoHcy-6-azide (isomer II) versus ETA-AdoHcy-6-azide (isomer II) with M.Mpel enzyme and pUC19 plasmid DNA. AdoHcy-6-azide and ETA-AdoHcy-6-azide concentrations were 250-31.25 UM, Lanes 1-4: serial dilutions of AdoHcy-6-azide; Lane 5: control of restriction enzyme-PUC19 fully digested in presence of AdoHcy-6-azide (250 μM); Lanes 6-9: serial dilutions of ETA-AdoHcy-6-azide; Lane 10: control of restriction enzyme-pUC19 fully digested in presence of ETA-AdoHcy-6-azide (250 μM); Lanes 11-12: positive control with AdoMet of complete protection; Lane 13: negative control with no cofactor; Lane 14: negative control with no enzyme and cofactor.

The bands located closer to the top of the image correspond to larger fragments of DNA whereas bands located towards the bottom of the image correspond to smaller DNA fragments, indicating more digestion by the restriction enzyme.

The pUC19 circular plasmid was treated with M.Mpel and ETA-AdoHcy-6-azide. Upon successful reaction cytosine residues within CG motifs are modified and hence protected from the restriction enzyme during the following step of the assay.

Lanes 6-9 (ETA-AdoHcy-6-azide isomer II) show more bands towards the top of the image (larger size of DNA) compared to respective lanes 1-4(AdoHcy-6-azide isomer II). This is a result of higher degree of protection of pUC19 DNA when ETA-AdoHcy-6-azide is used compared to AdoHcy-6-azide.

FIG. 3 shows the result of a protection assay of b-Ala-AdoHcy-6-azide (isomers I and II) with M.Mpel enzyme and pUC19 plasmid DNA. b-Ala-AdoHcy-6-azide concentrations were 250-31.25 μM. Lanes 1-4: serial dilutions of b-Ala-AdoHcy-6-azide (isomer I); Lane 5: control of restriction enzyme-pUC19 fully digested in presence of b-Ala-AdoHcy-6-azide (isomer I) (250 UM); Lanes 6-9: serial dilutions of b-Ala-AdoHcy-6-azide (isomer II); Lane 10: control of restriction enzyme-pUC19 fully digested in presence of b-Ala-AdoHcy-6-azide (isomerll) (250 UM); Lanes 11-12: positive control with AdoMet of complete protection; Lane 13: negative control with no cofactor; Lane 14: negative control with no enzyme and cofactor.

Lanes 6-9 (b-Ala-AdoHcy-6-azide (isomer II)) show more bands towards the top of the image (larger size of DNA) compared to lanes 1˜4 (b-Ala-AdoHcy-6-azide (isomer I)), indicating that isomer II results in a greater degree of modification, and therefore protection, of pUC19 DNA.

FIG. 4 shows the result of a protection assay of AdoHcy-6-azide (isomer II) versus b-Ala-AdoHcy-6-azide (isomer II) with M.Mpel enzyme and pUC19 plasmid DNA. AdoHcy-6-azide and b-Ala-AdoHcy-6-azide concentrations were 250-31.25 μM. Lanes 1-4: serial dilutions of AdoHcy-6-azide; Lane 5: control of restriction enzyme-PUC19 fully digested in presence of AdoHcy-6-azide (250 μM); Lanes 6-9: serial dilutions of b-Ala-AdoHcy-6-azide; Lane 10: control of restriction enzyme-pUC19 fully digested in presence of b-Ala-AdoHcy-6-azide (250 M); Lanes 11-12: positive control with AdoMet of complete protection; Lane 13: negative control with no cofactor; Lane 14: negative control with no enzyme and cofactor.

We surprisingly found that where R₃ is a short, flexible alkyl chain (C₁-C₄) that sits within the cofactor binding pocket of the enzyme and is terminated with an electron-rich, hydrogen bond accepting group, the activity of the enzyme (M.Mpel)-cofactor pair improved relative to the unmodified (R₂═R₃═H) cofactor analogue. Indeed, in the case where R₃ is C₂ alkyl and R₄ is —OH (FIG. 2) we found similar DNA alkylation efficiencies using around half the amount of cofactor. Further, the extent of DNA alkylation could be driven towards higher overall efficiencies than was possible with the unmodified (R₂═R₃═H) cofactor analogue. In the case where R₃ is C₂ alkyl and R₄ is —C(O) OH (FIGS. 3, 4) we also noted similar enhancements in overall DNA transalkylation efficiency. As a result, we would expect to see similar behaviour from cofactors carrying a modification at R₂ or R₃ that enables hydrogen bonding to an available amino acid in the cofactor binding pocket of a given methyltransferase enzyme. Such groups will typically have short, flexible alkyl chains and a small terminal functional group that can act as a hydrogen bond acceptor at neutral pH (such as alcohol, carboxylic acid, aldehyde, ketone, ester, amide, thiol).

### Example 4: Methods Utilising Compounds of Formula (I)

### Fluorescent Labelling of Genomic DNA/Optical Mapping

Fluorescent DNA labelling can be combined with linearisation of long (up to hundreds of kilobase pairs) genomic DNA molecules in order to visualise the labelling pattern on the DNA sequence, that is introduced by a given methyltransferase.

A 200 μl solution containing 1x CutSmart Buffer (NEB), 10 μg genomic DNA, 0.9 μg Taql DNA methyltransferase (M.Taql) and 750 UM cofactor analogue was prepared and incubated at 50° C. for 1 hour. Subsequently, 5 μl 18 mg/ml proteinase K (NEB)/0.1% Triton X-100 (Sigma-Aldrich) was added and this was incubated at 50° C. for 1 hour, before purification by GenElute Bacterial Genomic DNA kit (Sigma-Aldrich). DNA was eluted into 200 μl TE Buffer (10 mM tris, 1 mM EDTA). Meanwhile, a 20 μl solution containing 0.5× phosphate buffered saline (Sigma-Aldrich), 10 μl DMSO, 1 mM dibenzylcyclooctyne-amine (Sigma-Aldrich) and 12.5 mM Atto 647N-NHS ester (Sigma-Aldrich) was incubated at 4° C. for 1 hour. The DNA sample was split into 30 μl aliquots and 10 μl of the mixture containing the Atto 647N was added to an aliquot. This mixture was incubated at room temperature overnight, before purification by GenElute Bacterial Genomic DNA kit and eluted into 50 μl TE Buffer (10 mM tris, 1 mM EDTA).

### Molecular Combing

Molecular combing of DNA was performed based on the procedure described by Deen et al. (Deen,J., Sempels,W., De Dier,R., Vermant,J., Dedecker, P., Hofkens,J. and Neely, R.K. (2015) Combing of Genomic DNA from Droplets Containing Picograms of Material. ACS Nano, 9, 809-816.). Glass coverslips (Borosilicate Glass No. 1, Thermo Fisher) were cleaned to remove any fluorescent contaminants by incubation in a furnace oven at 450° C. for 24 hours. After removing from the furnace and allowing to cool, 30 μl of Zeonex solution (Zeon Chemicals, 1.5% w/v solution Zeonex 330R in chlorobenzene) was deposited onto a coverslip on a spin coater (Ossila) and subsequently spun at 3000 rpm for 90 seconds. Zeonex-coated coverslips were allowed to dry at room temperature overnight and stored in a desiccator.

To perform the molecular combing, 2 μl Atto 647N-labelled DNA (prepared as described above) (2 ng/μl in 1xTE) was suspended in 17 μl 100 mM sodium phosphate buffer (pH 5.7) containing 1 μl DMSO. A 1.5 μl droplet of this solution was deposited on the surface of the Zeonex-coated coverslip. A clean pipette tip was placed in contact with the droplet and used to drag it, with a velocity of approximately 5 mm/min, across the coverslip. Fluorescence microscopy

Deposited DNA was imaged on an ASI RAMM microscope, equipped with a Nikon 100× TIRF objective. Illumination was from a 100 mW OBIS 640 nm CW laser via a quad-band dichroic mirror (405/488/561/635) and images were collected using an Evolve Delta EM-CCD camera, via a quad-band emission filter (Semrock, 432/515/595/730 nm). Micromanager was used to control the system and scan the sample (17).

### DNA Barcode Extraction, Pairwise Alignment and Community Detection

Software was written in MATLAB (R2016b, The MathWorks, Inc., Natick, Massachusetts, United States of America) for the automated extraction of DNA barcodes from microscopy images, in silico generation of DNA barcodes and the alignment procedures.

Although the above examples demonstrate the interaction between the AdoMet analogues and plasmid DNA it will be appreciated that other biomolecules (RNA, proteins, small molecules etc) can be alkylated using the AdoMet analogues of formula (I) and the appropriate methyltransferase (for example see Angew. Chem. Ind. 2017 (56) 5182-5200.

1. An AdoMet analogue of formula (I), 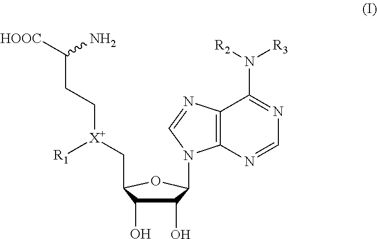
wherein:
X is S or Se;
R₁ has the structure [R₅]_{q}—[L₁]ₚ—[HM]ₙ—[L₂]ₘ—U—CH₂—;
R₂ is H and R₃ is (C₁-C₄)alkyl, (C₂-C₄)alkenyl or (C₂-C₄)alkynyl, with the proviso that R₃ is not propargyl, optionally wherein R₃ is substituted with one or more R₄, or
R₂ and R₃, together with the nitrogen to which they are attached, form a 5- or 6-membered heterocyclyl ring which is optionally substituted with one or more R₄;
R₄ is selected from the group consisting of: —NRₐR_{b}; —OH; —SH; —CN; —C(O) OR₆; —C(O) R₆;
C(O) NRₐR_{b}; N₃; and halo;
R₆ is H or unsubstituted C₁-C₄ alkyl;
Rₐ and R_{b} are independently selected from H and unsubstituted C₁-C₄ alkyl;
L₁ is a bond or a linker;
HM is a hydrolysable moiety;
L₂ is a linker;
U comprises an unsaturated group selected from an alkene, an alkyne, an aromatic group, a carbonyl group, and a sulphur atom comprising one or two S═O bonds;
m, n, p and q are each independently selected from 0 and 1;
R₅ comprises a heavy atom or a heavy atom cluster suitable for phasing of X-ray diffraction data, a radioactive or stable rare isotope, a fluorophore, a fluorescence quencher, an affinity tag, a crosslinking agent, a nucleic acid cleaving reagent, a spin label, a chromophore, a protein, peptide or amino acid which may optionally be modified, a nucleotide, nucleoside or nucleic acid which may optionally be modified, a carbohydrate, a lipid, a transfection reagent, an intercalating agent, a nanoparticle or bead, or a functional group,
wherein the functional group is selected from the group consisting of: an unprotected amino group a protected amino group, a thiol group, a 1,2-diol group, a hydrazino group, a hydroxyamino group, a haloacetamide group, a maleimide group, a cyanide group, a cyclic hydrocarbon a halo group, an aldehyde group, a ketone group, a 1,2-aminothiol group, a azido group, an isothiocyanate group, a thiocyanate group, an alkene group, an alkyne group, 1,3-diene function, a dienophilic function, an arylhalide group, an arylboronic acid group, a terminal haloalkyne group, a terminal silylalkyne group, —N═C═O; —N═C═S, —O—C(O)NH₂, a group comprising a sterically strained alkyne or alkene, a nitrone, a tetrazine, a tetrazole, and a 1,2-aminothiol group.

2. The AdoMet analogue of claim 1, wherein R₅ is selected from the group consisting of: halo (—F, —Cl, —Br, —I); —C═C; —C═C; —N₃; —N═C═O; —N═C═S; —O—C(O)NH₂; —SH, epoxide; —NH₂; —CEN; a nitrone, a tetrazine, a tetrazole, a sterically strained alkyne or a sterically strained alkene.

3. The AdoMet analogue of claim 1, wherein L₁ is a linker comprising a linear chain of from 1 to 50 atoms, optionally wherein L₁ comprises a hydrocarbon and/or a polyether chain.

4. The AdoMet analogue of claim 3, wherein L₁ comprises a polyethylene glycol chain comprising up to 15 monomers of ethylene glycol.

5. The AdoMet analogue of claim 4, wherein L₁ has the structure: 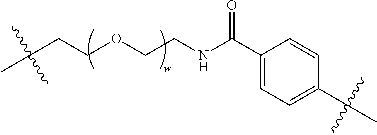
wherein w is an integer of from 1 to 15.

6. The AdoMet analogue of any-preceding claim 1, wherein the hydrolysable moiety HM is selected from the group consisting of: 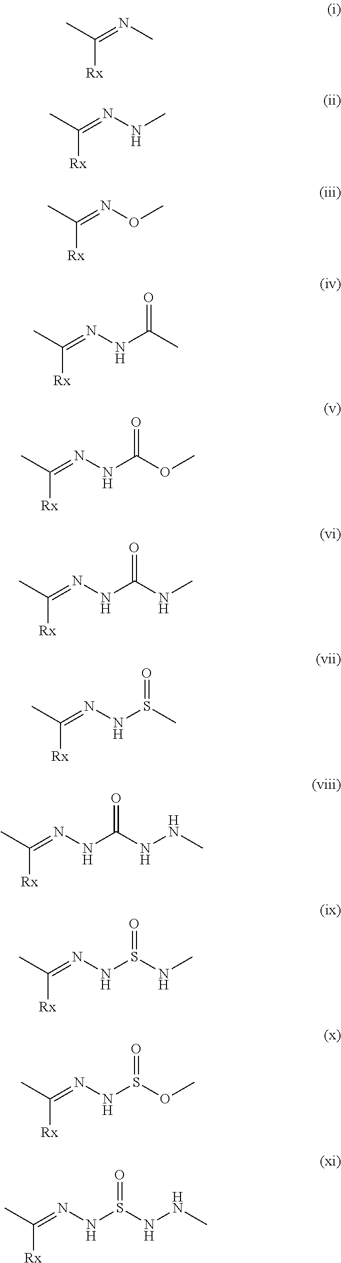 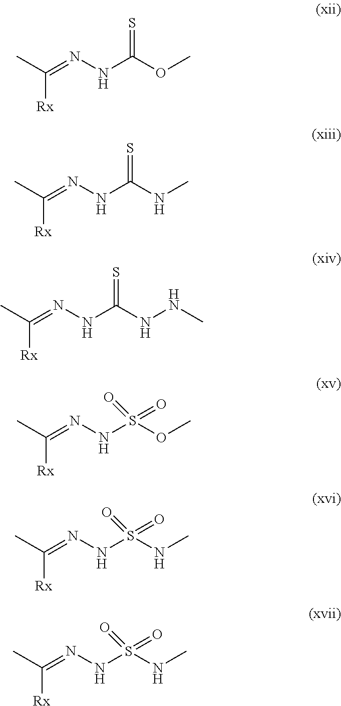
wherein Rx is selected from: a hydrogen atom, a deuterium atom and unsubstituted C₁-C₄ alkyl, optionally wherein the hydrolysable moiety HM has the structure:

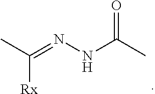

7. The AdoMet analogue of claim 1, wherein L₂ comprises a linear chain of from 1 to 20 atoms, optionally wherein L₂ comprises a linear C₁-C₁₀ alkyl chain, further optionally wherein the alkyl chain is unsubstituted.

8. The compound of claim 1, wherein R₁ has the structure:

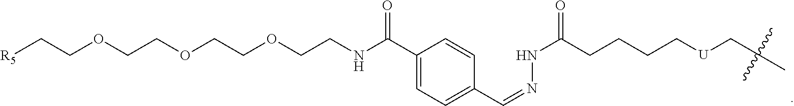

9. The AdoMet analogue of claim 1, wherein L₁ comprises a linear C₁-C₁₀ alkyl chain, optionally wherein the alkyl chain is unsubstituted.

10. The AdoMet analogue of claim 9, wherein R₁ has the structure:

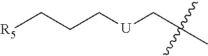

11. The AdoMet analogue of claim 1, wherein R₅ is N₃ and/or wherein U is —C═C—.

12. The AdoMet analogue of claim 1, wherein R₁ has the structure:
R₅-L₁-U—
wherein R₅, L₁ and U are as defined above.

13. The AdoMet analogue of claim 1, wherein R₁ has the structure:

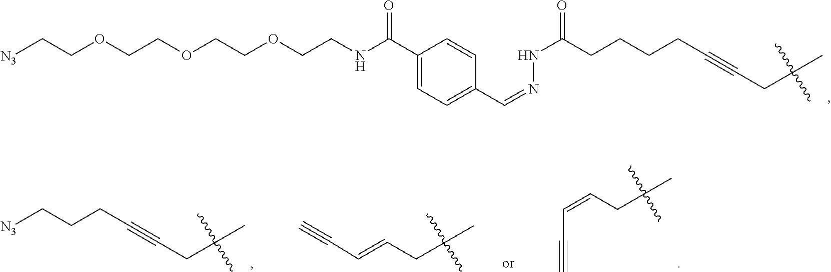

14. The AdoMet analogue of any preceding claim 1, wherein;
(i) R₂ is H and R₃ is (C₁-C₄)alkyl substituted with one or more R₄;
(ii) R₂ is H and R₃ is C₂ alkyl substituted with one or more R₄; or
(iii) R₂ is H and R₃ is selected from the group consisting of: 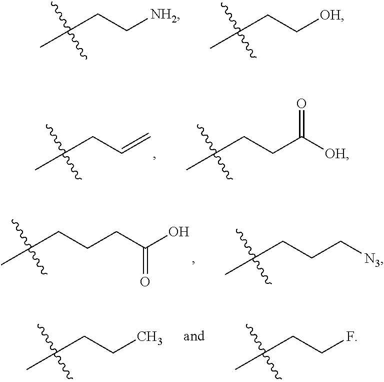
or wherein R₂ and R₃, together with the nitrogen to which they are attached, form the structure: 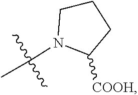 [SMILES: *N1CCCC1C(=O)O]
optionally wherein the structure is

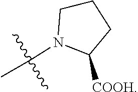 [SMILES: *N1CCC[C@H]1C(=O)O]

15. (canceled)

16. The AdoMet analogue of any preceding claim 1, wherein
(i) R₄ is selected from: —NRₐR_{b}; —OH; —SH; —C(O) OR₆; —C(O)R₆; and C(O)NRₐR_{b};
(ii) R₄ is —OH; or
(iii) R₄ is —C(O)OH.

17-19. (canceled)

20. The AdoMet analogue of claim 1, wherein X is S.

21. The AdoMet analogue of claim 1, wherein the compound is selected from:

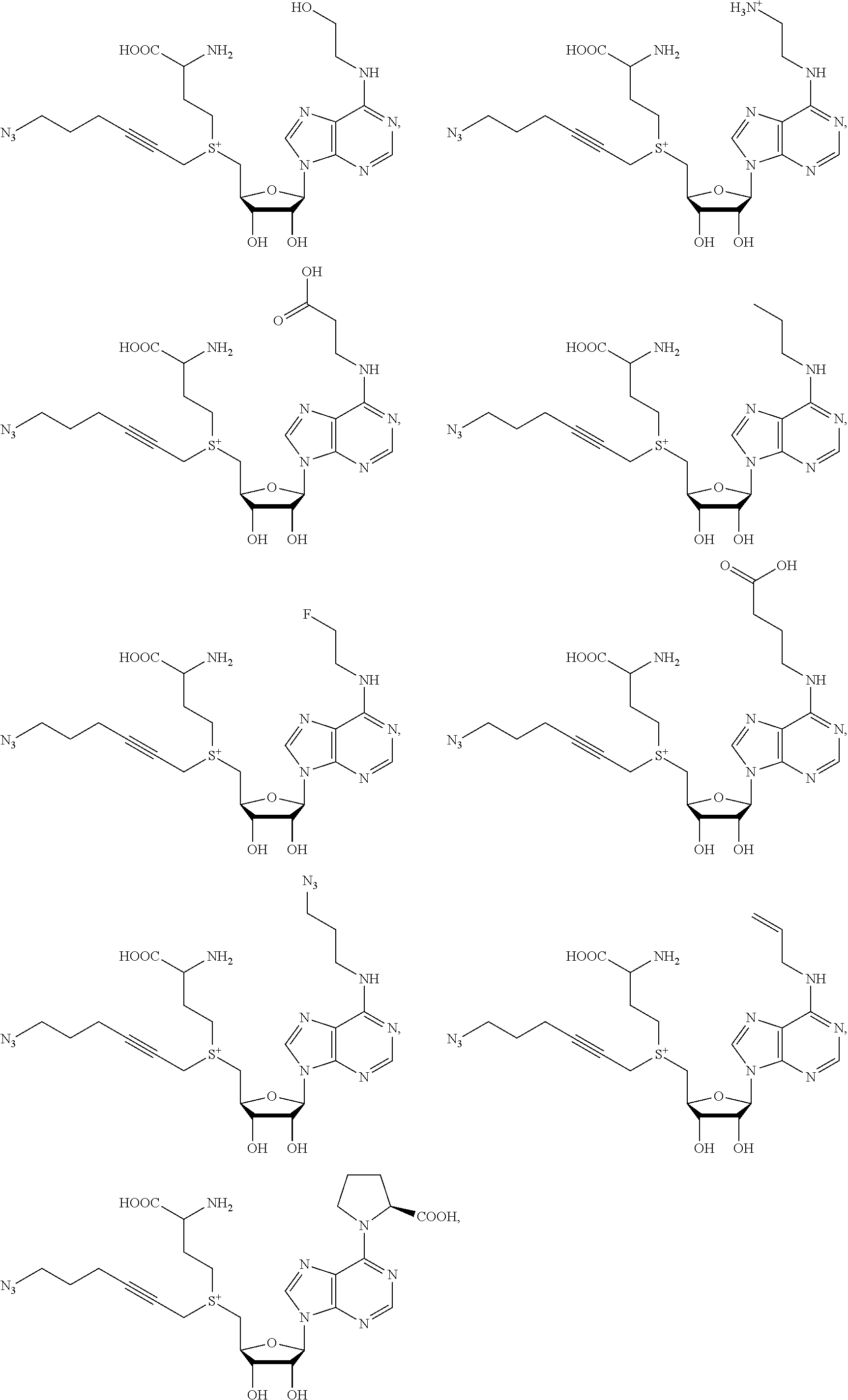

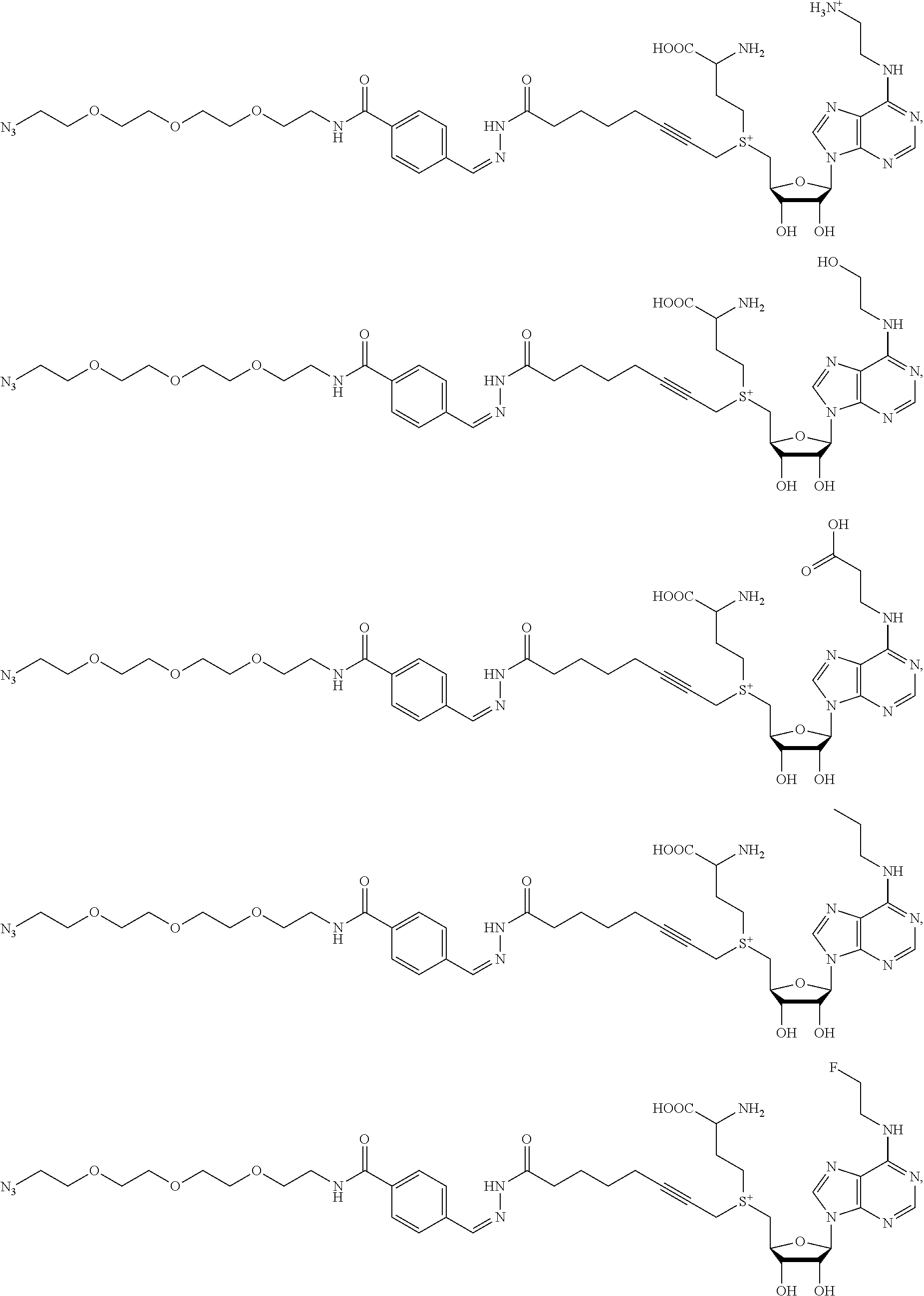

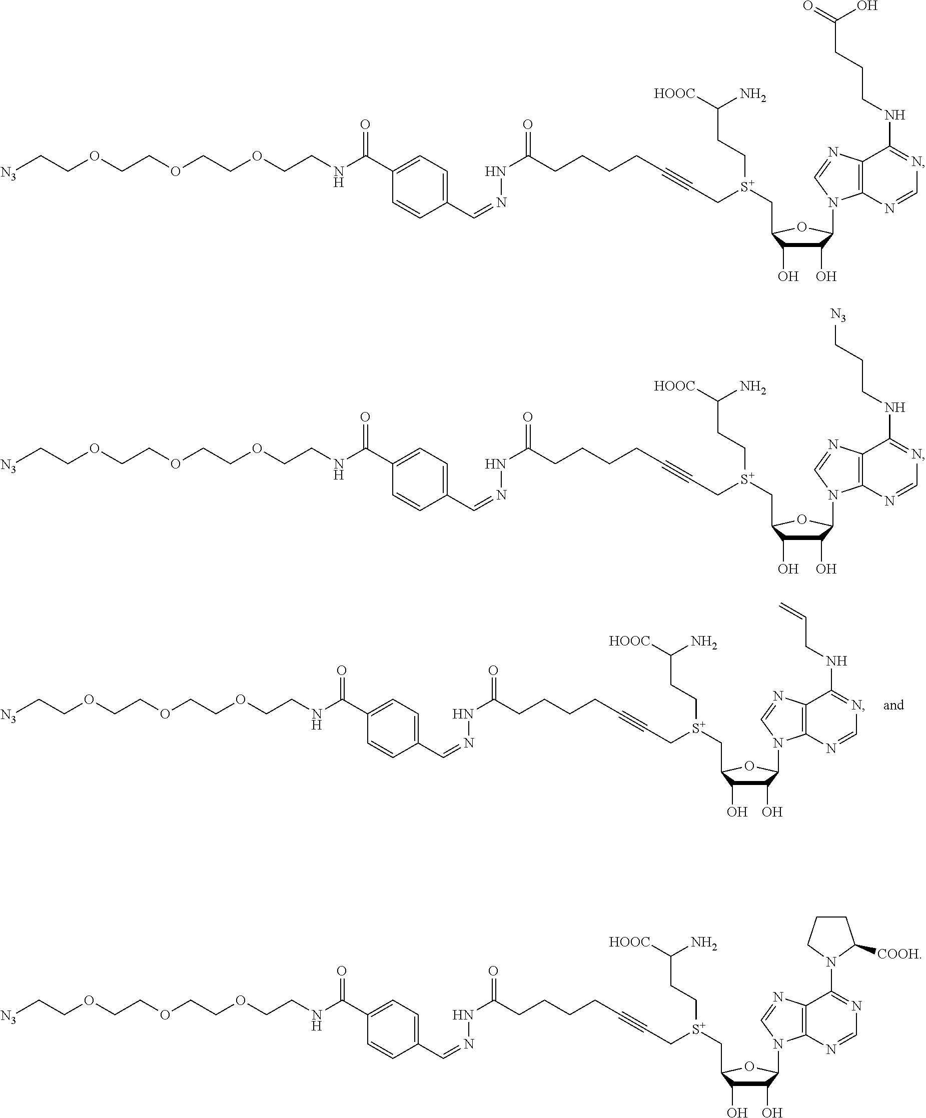

22-25. (canceled)

26. A method of modifying a target molecule, the method comprising incubating the target molecule with an AdoMet analogue according to claim 1 and a methyltransferase such that a part of said compound is transferred onto the target molecule.

27-29. (canceled)

30. A biomolecule having bonded
thereto a molecule R₁, wherein
R₁ has the structure [R₅]_{q}—[L₁]ₚ—[HM]ₙ—[L₂]ₘ—U—CH₂—
L₁ is a bond or a linker;
HM is a hydrolysable moiety;
L₂ is a linker;
U comprises an unsaturated group selected from an alkene, an alkyne, an aromatic group, a carbonyl group, and a sulphur atom comprising one or two S═O bonds;
m, n, p and q are each independently selected from 0 and 1; and
R₅ comprises a heavy atom or a heavy atom cluster suitable for phasing of X-ray diffraction data, a radioactive or stable rare isotope, a fluorophore, a fluorescence quencher, an affinity tag, a crosslinking agent, a nucleic acid cleaving reagent, a spin label, a chromophore, a protein, peptide or amino acid which may optionally be modified a nucleotide, nucleoside or nucleic acid which may optionally be modified, a carbohydrate, a lipid, a transfection reagent, an intercalating agent, a nanoparticle or bead, or a functional group,
wherein the functional group is selected from the group consisting of: an unprotected amino group, a protected amino group, a thiol group, a 1,2-diol group, a hydrazino group, a hydroxyamino group, a haloacetamide group, a maleimide group, a cyanide group, a cyclic hydrocarbon a halo group, an aldehyde group, a ketone group, a 1,2-aminothiol group, a azido group, an isothiocyanate group, a thiocyanate group, an alkene group, an alkyne group, a 1,3-diene function, a dienophilic function, an arylhalide group,
an arylboronic acid group, a terminal haloalkyne group, a terminal silylalkyne group, —N═C═O; —N═C═S, —O—C(O)NH₂, a group comprising a sterically strained alkyne or alkene, a nitrone, a tetrazine, a tetrazole, and 1,2-aminothiol group.
